SEQUENCE LISTING (1 sequence)

SEQ ID NO: 1 - protein, 8 aa, Artificial Sequence
  synthetic peptide
SIINFEKL

(19) United States
(12) Patent Application Publication
(10) Pub. No.: US 2023/0046425 A1
(43) Pub. Date: Feb. 16, 2023

(54) IMMUNOTHERAPY FOR DIRECT REPROGRAMMING OF CANCER CELLS INTO IMMUNE CELLS/ANTIGEN PRESENTING CELLS/DENDRITIC CELLS

(71) Applicant: University of Florida Research Foundation, Incorporated, Gainesville, FL (US)

(72) Inventors: David TRAN, Gainesville, FL (US); Son LE, Gainesville, FL (US)

(21) Appl. No.: 17/788,519

(22) PCT Filed: Dec. 22, 2020
(86) PCT No.: PCT/US2020/066557

§ 371 Date: Jun. 23, 2022

Related U.S. Application Data
(60) Provisional application No. 62/952,725, filed on Dec. 23, 2019.

(51) Int. Cl. A61K 31/7088; C12N 15/63; C12N 5/09; A61P 35/00

(52) U.S. Cl. CPC A61K 31/7088; C12N 15/63; C12N 5/0693; A61P 35/00

(57) ABSTRACT

Described are compounds and compositions for transdifferentiation of glioblastoma cells to antigen presenting cells. Methods of using the compounds and compositions to treat glioblastoma and to induce an immune response against a glioblastoma are also described.

### CROSS-REFERENCE TO RELATED APPLICATIONS

This application claims the benefit of U.S. Provisional Application No. 62/952,725, filed Dec. 23, 2019, which is incorporated herein by reference.

### SEQUENCE LISTING

The Sequence Listing written in file 554362 SeqListing.txt is 1 kilobytes in size, was created Dec. 8, 2020, and is hereby incorporated by reference.

### BACKGROUND

Glioblastoma (GBM) is the most common and lethal malignant brain cancer in adults. Despite aggressive treatment, the 5-year survival rate remains <5%. Like in other cancers, immunotherapy has emerged as a potentially powerful approach to achieve long-term survival in patients with GBM.

Dendritic cells (DC) play a central role in priming cancer-specific immune responses due to their ability to sample and present tumor antigens and neoantigens to the immune system and are currently undergoing clinical trials with promising results in GBM. However, one of the limiting factors in the DC immunotherapy approach in GBM is the inefficient migration of DC to brain tumors. Other general limitations include the difficulty with isolation and generation of effective DC and the high cost of cell-based immunotherapy.

Recent advances in treatment for patients with glioblastoma (GBM) have produced only a modest survival benefit with few long-term survivors. New effective, and safe therapies are urgently needed to enhance outcomes for GBM patients.

Therefore, there is an urgent need for innovative therapeutic approaches for GBM, especially in immunotherapy.

### SUMMARY

Described are methods of treating glioblastoma by administering to a glioblastoma one or more nucleic acids that increase and/or decrease expression of one or more glioblastoma to antigen presenting cell transdifferentiation determinants. A nucleic acid that increases expression of a transdifferentiation determinant can be a nucleic acid encoding a positive regulator of transdifferentiation. The positive regulator, when expressed in a cell of the glioblastoma, facilitates transdifferentiation of the glioblastoma cell into an antigen presenting cell. A nucleic acid that decreases expression of a transdifferentiation determinant can be an expression inhibitor that inhibits expression of a negative regulator of transdifferentiation. The expression inhibitor, when delivered to a cell of the glioblastoma, inhibits expression of a negative regulator of transdifferentiation and facilitates transdifferentiation of the glioblastoma cell into an antigen presenting cell. The glioblastoma cell can be transdifferentiated into a dendritic-like cell or a macrophage-like cell. A positive regulator of transdifferentiation, when expressed in a glioblastoma cell can also reduce growth of the glioblastoma cell. The glioblastoma can be a mammalian glioblastoma, such as, but not limited to, a human glioblastoma or a mouse glioblastoma. In some embodiments, the glioblastoma is in the brain. In some embodiments, the glioblastoma is in the spinal cord. In some embodiments, the glioblastoma cell is a cancer stem cell (CSC).

Described are methods of treating high grade glioma (WHO grade III or IV) by administering to a high-grade glioma one or more nucleic acids that increase and/or decrease expression of one or more glioblastoma to antigen presenting cell transdifferentiation determinants. A nucleic acid that increases expression of a transdifferentiation determinant can be a nucleic acid encoding a positive regulator of transdifferentiation. The positive regulator, when expressed in a cell of the glioblastoma, facilitates transdifferentiation of the glioblastoma cell into an antigen presenting cell. A nucleic acid that decreases expression of a transdifferentiation determinant can be an expression inhibitor that inhibits expression of a negative regulator of transdifferentiation. The expression inhibitor, when delivered to a cell of the glioblastoma, inhibits expression of a negative regulator of transdifferentiation and facilitates transdifferentiation of the glioblastoma cell into an antigen presenting cell. The high-grade glioma cell can be transdifferentiated into a dendritic-like cell or a macrophage-like cell. A positive regulator of transdifferentiation, when expressed in a high-grade glioma cell can also reduce growth of the high-grade glioma cell. The high-grade glioma can be a mammalian high grade glioma, such as, but not limited to, a human high-grade glioma or a mouse high grade glioma. In some embodiments, the high-grade glioma is in the brain. In some embodiments, the high-grade glioma is in the spinal cord. In some embodiments, the glioblastoma cell is a CSC.

Described are methods of treating glioblastoma multiforme (GBM) by administering to a GBM one or more nucleic acids that increase and/or decrease expression of one or more glioblastoma to antigen presenting cell transdifferentiation determinants. A nucleic acid that increases expression of a transdifferentiation determinant can be a nucleic acid encoding a positive regulator of transdifferentiation. The positive regulator, when expressed in a cell of the glioblastoma, facilitates transdifferentiation of the glioblastoma cell into an antigen presenting cell. A nucleic acid that decreases expression of a transdifferentiation determinant can be an expression inhibitor that inhibits expression of a negative regulator of transdifferentiation. The expression inhibitor, when delivered to a cell of the glioblastoma, inhibits expression of a negative regulator of transdifferentiation and facilitates transdifferentiation of the glioblastoma cell into an antigen presenting cell. The GBM cell can be transdifferentiated into a dendritic-like cell or a macrophage-like cell. A positive regulator of transdifferentiation, when expressed in a GBM cell can also reduce growth of the GBM cell. The GBM can be a mammalian GBM, such as, but not limited to, a human GBM or a mouse GBM. In some embodiments, the GBM is in the brain. In some embodiments, the GBM is in the spinal cord. In some embodiments, the glioblastoma cell is a CSC.

Described are methods of inducing an immune response to a glioblastoma, including a high-grade glioma or a GBM, comprising, administering to a glioblastoma one or more nucleic acids that increase and/or decrease expression of one or more glioblastoma to antigen presenting cell transdifferentiation determinants. A nucleic acid that increases expression of a transdifferentiation determinant can be a nucleic acid encoding a positive regulator of transdifferentiation. The positive regulator, when expressed in a cell of the glioblastoma, facilitates transdifferentiation of the glioblastoma cell into an antigen presenting cell, such as a dendritic-like cell or a macrophage-like cell. A nucleic acid that decreases expression of a transdifferentiation determinant can be an expression inhibitor that inhibits expression of a negative regulator of transdifferentiation. The expression inhibitor, when delivered to a cell of the glioblastoma, inhibits expression of a negative regulator of transdifferentiation and facilitates transdifferentiation of the glioblastoma cell into an antigen presenting cell, such as a dendritic-like cell or a macrophage-like cell. The antigen presenting cell can then present glioblastoma antigen(s) or neoantigen(s) to immune cells, thereby stimulating an immune response against the glioblastoma. The glioblastoma can be a mammalian glioblastoma, such as, but not limited to, a human glioblastoma or a mouse glioblastoma. In some embodiments, the glioblastoma is in the brain. In some embodiments, the glioblastoma is in the spinal cord. In some embodiments, the glioblastoma is a high-grade glioma. In some embodiments, the glioblastoma is a GBM. In some embodiments, the glioblastoma cell is a CSC.

Also described are methods for transdifferentiation of glioblastoma cells into antigen presenting cells comprising: delivering to the glioblastoma cells one or more nucleic acids that increase and/or decrease expression of one or more glioblastoma to antigen presenting cell transdifferentiation determinants. A nucleic acid that increases expression of a transdifferentiation determinant can be a nucleic acid encoding a positive regulator of transdifferentiation. The positive regulator, when expressed in a cell of the glioblastoma, facilitates transdifferentiation of the glioblastoma cell into an antigen presenting cell, such as a dendritic-like cell or a macrophage-like cell. A nucleic acid that decreases expression of a transdifferentiation determinant can be an expression inhibitor that inhibits expression of a negative regulator of transdifferentiation. The expression inhibitor, when delivered to a cell of the glioblastoma inhibits expression of a negative regulator of transdifferentiation and facilitates transdifferentiation of the glioblastoma cell into an antigen presenting cell, such as a dendritic-like cell or a macrophage-like cell. The transdifferentiated glioblastoma cell can be a dendritic-like cell or a macrophage-like cell. The glioblastoma cell can be a mammalian glioblastoma cell, such as, but not limited to, a human glioblastoma cell or a mouse glioblastoma cell. In some embodiments, the glioblastoma is a high-grade glioma. In some embodiments, the glioblastoma is a GBM.

In some embodiments, two, three, four, or more nucleic acids that increase and/or decrease expression of one or more glioblastoma to antigen presenting cell transdifferentiation determinants are delivered to the glioblastoma or the glioblastoma cells. In some embodiments, two, three, four, or more nucleic acids that increase and/or decrease expression of one or more transdifferentiation determinants are delivered to the high-grade glioma or the high grade glioma cells. In some embodiments, two, three, four, or more nucleic acids that increase and/or decrease expression of one or more transdifferentiation determinants are delivered to the GBM or the GBM cells. For administration of two of more nucleic acids that increase and/or decrease expression of one or more transdifferentiation determinants, the nucleic acids may be administered simultaneously or sequentially. For sequential administration, the two different nucleic acid may be administered to the glioblastoma 1, 2, 3, 4, 5, 6, 7, 8, 9, 10, 11, 12, 13, or 14 days apart.

In some embodiments, the glioblastoma or glioblastoma cell is a human glioblastoma or human glioblastoma cell, and the transdifferentiation determinant is a human gene. In some embodiments, the glioblastoma is a high-grade glioma. In some embodiments, the glioblastoma is a GBM. In some embodiments, the glioblastoma cell is a cancer stem cell. In some embodiments, the human gene transdifferentiation determinant is selected from Table 1. In some embodiments, a human gene transdifferentiation determinant is selected from the group consisting of: SPI1, IKZF1, ARID3A, ATF5, BASP1, BCL6, CIITA, CIR1, CREG1, CTSZ, ELF4, FOXP1, GNS, HHEX, HTATIP2, IRF5, KLF11, KMT2E, LDB1, LMO2, MMP14, MREG, MXD1, NAB2, NCOA3, NFE2L1, NOTCH2, NR1H3, PRKCB, SATB1, STAT6, TDP2, TFEC, USF1, USF2, ZBTB34, ZFP91, ZNF366. In some embodiments, increasing and/or decreasing expression of the one or more transdifferentiation determinants can be used to transdifferentiate a glioblastoma cancer stem cell (CSC) to an antigen presenting cell. In some embodiments, increasing and/or decreasing expression of the one or more transdifferentiation determinants can be used to transdifferentiate a glioblastoma cell to an antigen presenting cell. The antigen presenting cell can be, e.g., a dendritic-like cell or a macrophage-like cell.

In some embodiments, increasing and/or decreasing expression of the one or more transdifferentiation determinants is used to transdifferentiate a glioblastoma cell to a dendritic-like cell. In some embodiments, the glioblastoma or glioblastoma cell is a mouse glioblastoma or mouse glioblastoma cell, and the transdifferentiation determinant is a mouse gene. In some embodiments, the mouse gene transdifferentiation determinant is selected from the group consisting of: IRF5, CBFA2T3, IRF8, ATOX1, SPI1, BCL11A, ID2, MYCL, BATF3, HHEX, SPIB, VAV1, ETV6, MXD1, ETV3, LMO2, AES, GLMP, CEBPA, and MAZ. In some embodiments, the glioblastoma or glioblastoma cell is a human GBM cell or a human GBM-CSC, and the transdifferentiation determinant is a human gene. In some embodiments, the human gene transdifferentiation determinant is selected from the group consisting of: SPI1, IKZF1, FOXP1, CTSZ, HHEX, TFEC, HTATIP2, KLF11, PRKCB, USF1, TDP2, BCL6, CREG1, ZNF366, LDB1, KMT2E, CIITA, ZFP91, ELF4, NCOA3, ZBTB34, ARID3A, SATB1, STAT6, LMO2, and NAB2.

In some embodiments, increasing and/or decreasing expression of the one or more the one or more transdifferentiation determinants is used to transdifferentiate a glioblastoma cell to a macrophage-like cell. In some embodiments, the glioblastoma or glioblastoma cell is a mouse glioblastoma or mouse glioblastoma cell, and the transdifferentiation determinant is a mouse gene. In some embodiments, the mouse gene transdifferentiation determinant is selected from the group consisting of: SPI1, IRF5, IRF8, TFE3, CEBPA, BCL11A, CEBPB, SPIB, POU2AF1, HELZ2, IKZF3, MAFB, LMO2, VAV1, ARF2, CBFA2T3, MAZ, PPARD, TAF10, and ZFP384. In some embodiments, the glioblastoma or glioblastoma cell is a human GBM cell or a human GBM-CSC, and the transdifferentiation determinant is a human gene. In some embodiments, the human gene transdifferentiation determinant is selected from the group consisting of: SPI1, IKZF1, CTSZ, TFEC, HTATIP2, FOXP1, CREG1, TDP2, PRKCB, CIR1, NR1H3, KLF11, GNS, MMP14, HHEX, BASP1, KMT2E, ATF5, NFE2L1, IRF5, SATB1, ARID3A, ZBTB34, NOTCH2, MXD1, USF2, MREG.

In some embodiments, at least two different nucleic acids for increasing and/or decreasing expression at least two different transdifferentiation determinants are administered to the glioblastoma or glioblastoma cells. In some embodiments, at least one of the at least two different transdifferentiation determinants comprises SPI1 or IKZF1. In some embodiments, at least one of the at least two different transdifferentiation determinants comprises SPI1. In some embodiments, at least one of the at least two different transdifferentiation determinants comprises IKZF1. In some embodiments, the second transdifferentiation determinant can be selected from the group consisting of: SPI1 (when the first determinant is IKZF1), IKZF1 (when the first determinant is SPI1), ARID3A, ATF5, BASP1, BCL6, CIITA, CIR1, CREG1, CTSZ, ELF4, FOXP1, GNS, HHEX, HTATIP2, IRF5, KLF11, KMT2E, LDB1, LMO2, MMP14, MREG, MXD1, NAB2, NCOA3, NFE2L1, NOTCH2, NR1H3, PRKCB, SATB1, STAT6, TDP2, TFEC, USF1, USF2, ZBTB34, ZFP91, ZNF366. In some embodiments, the at least two different transdifferentiation determinants comprise SPI1 and IKZF1. In some embodiments, the at least two different transdifferentiation determinants further comprises a third transdifferentiation determinant. The third transdifferentiation determinant can be selected from the group consisting of: SPI1 (when neither the first nor the second determinant is SPI1), IKZF1 (when neither the first nor the second determinant is IKZF1), ARID3A, ATF5, BASP1, BCL6, CIITA, CIR1, CREG1, CTSZ, ELF4, FOXP1, GNS, HHEX, HTATIP2, IRF5, KLF11, KMT2E, LDB1, LMO2, MMP14, MREG, MXD1, NAB2, NCOA3, NFE2L1, NOTCH2, NR1H3, PRKCB, SATB1, STAT6, TDP2, TFEC, USF1, USF2, ZBTB34, ZFP91, ZNF366. In some embodiments, the three transdifferentiation determinants include SP1, IKZF1, and a third transdifferentiation determinant selected from the group consisting of: CTSZ, HTATIP2, TFEC, PRKCB, TDP2, CREG1, FOXP1, HHEX, KMT2E, USF1, ARID3A, CIITA, CIR1, GNS, KLF11, MMP14, NFE2L1, NR1H3, SATB1, ZBTB34, and ZNF366. In some embodiments, the three transdifferentiation determinants include SP1, IKZF1, and a third transdifferentiation determinant selected from the group consisting of: CTSZ, HTATIP2, TFEC, PRKCB, TDP2, CREG1, FOXP1, HHEX, and KMT2E. In some embodiments, the three transdifferentiation determinants include SP1, IKZF1, and a third transdifferentiation determinant selected from the group consisting of: CTSZ, HTATIP2, TFEC, PRKCB, TDP2, and CREG1.

In some embodiments, at least three different nucleic acids for increasing and/or decreasing expression at least three different transdifferentiation determinants are administered to the glioblastoma or glioblastoma cells. In some embodiments, at least one of the at least three different transdifferentiation determinants comprises SPI1 or IKZF1. In some embodiments, at least one of the at least three different transdifferentiation determinants comprises SPI1. In some embodiments, at least one of the at least three different transdifferentiation determinants comprises IKZF1. In some embodiments, at least two of the at least three different transdifferentiation determinants comprise SPI1 and IKZF1.

In some embodiments, the at least three transdifferentiation determinants comprise a first transdifferentiation determinant, a second transdifferentiation determinant, and a third transdifferentiation determinant, wherein the first transdifferentiation determinant comprises SPI1, the second transdifferentiation determinant comprises IKZF1, and the third transdifferentiation determinant is selected from the group consisting of: CTSZ, HTATIP2, TFEC, PRKCB, TDP2, CREG1, FOXP1, HHEX, KMT2E, USF1, ARID3A, CIITA, CIR1, GNS, KLF11, MMP14, NFE2L1, NR1H3, SATB1, ZBTB34, ZNF366. In some embodiments, the third transdifferentiation determinant is selected from the group consisting of: CTSZ, HTATIP2, TFEC, PRKCB, TDP2, CREG1, FOXP1, HHEX, KMT2E. In some embodiments, the third transdifferentiation determinant is selected from the group consisting of: CTSZ, HTATIP2, TFEC, PRKCB, TDP2, CREG1.

In some embodiments, the first transdifferentiation determinant comprises SP1, the second transdifferentiation determinant comprises IRF8, and the third transdifferentiation determinant comprises BATF3.

In some embodiments, the at least three different transdifferentiation determinants further comprises a fourth transdifferentiation determinant. In some embodiments, the fourth transdifferentiation determinant comprises ID2.

In some embodiments, the first transdifferentiation determinant comprises SPI1, the second transdifferentiation determinant comprises ID2, and the third transdifferentiation determinant comprises ATOX1. In some embodiments, the combination of SPI1, ID2, and ATOX1 further comprises a fourth transdifferentiation determinant comprising BCL11A.

In some embodiments, at least one of the nucleic acids for increasing and/or decreasing expression of a transdifferentiation determinant comprises an expression inhibitor for inhibiting expression of at least one negative regulator of transdifferentiation in the glioblastoma. The expression inhibitor can be, but is not limited to, an RNA interfering agent, such as an siRNA, or an antisense agent. In some embodiments, the expression inhibitor reduces expression of a gene in Table 8. In some embodiments two of more expression inhibitors are administered to a glioblastoma to inhibit expression of two or more transdifferentiation determinants.

In some embodiments, one or more genes encoding one or more positive regulators of transdifferentiation and one or more expression inhibitors for inhibiting expression of one or more negative regulators of transdifferentiation are administered to a glioblastoma to transdifferentiate cells of the glioblastoma to antigen presenting cells. The one or more genes encoding the one or more positive regulators can be selected from Tables 1-7. The one or more expression inhibitors can inhibit expression of one or more negative regulators selected from Tables 6 and 8.

### BRIEF DESCRIPTION OF THE FIGURES

Having thus described the invention in general terms, reference will now be made to the accompanying drawings, which are not necessarily drawn to scale.

FIG. 1. Graph illustrating efficient transdifferentiation of GBM to immune cells/DC-like cells by SPI1 (detaPEST)+IRF8 (18)+BATF3 (B2) or SPI1+IRF8+BATF3+ID2 (I) compared to the empty virus control (pSF).

FIG. 2. Graph illustrating reduced cellular growth rate in GBM cells expressing SPI1 (detaPEST)+IRF8 (18)+BATF3 (B3), or SPI1+IRF8+BATF3+ID2 (I). pSF=control vector.

FIG. 3. Graphs illustrating increased expression of CD11c, MHCII, and MHCI in cells expressing SPI1+IRF8+BATF3+ID2 (right curve in each panel), compared to empty vehicle (left curve in each panel).

FIG. 4A. Micrographs showing morphology (top two rows) or phagocytosis of pHrodo red Zymosan fungal particles (bottom row) of GBM cells treated with (a) SPI1+ID2+BATF3+IRF8 (4F, left panel) or (b) GBM cells treated with empty vector (middle panel); or (c) control dendritic cells (DC2.4, right panel). Arrows indicate presence of dendrites.

FIG. 4B. Graph illustrating expression of IL6 and TNFa in CD45⁻MHCII⁻ cells, CD45⁻MHCII⁺ cells, and CD45⁺MHCII⁺ cells.

FIG. 5. Graph illustrating terminal differentiation of transdifferentiated GBM cells (SldBI=SPI1+ID2+BATF3+IRF8).

FIG. 6A. Graph illustrating expression of antigen processing and presentation machinery components in GBM cells transduced with dP, m4F, or control vehicle.

FIG. 6B. Graph illustrating expression of dendritic cell-specific cytokines in GBM cells transduced with dP, m4F, or control vehicle.

FIG. 6C. Graph illustrating the percent of early activated CD69⁺CD4⁺ T cells (left panel) or early activated CD69⁺CD8⁺ T cells in GBM cells transduced with dP, m4F, or control vehicle.

FIG. 6D. Graph illustrating the IFN-γ production as determined by antigen (SIINFEKL (SEQ ID NO: 1))-specific T cell activation in GBM cells transduced with dP, m4F, or control vehicle.

FIG. 7A. Graph illustrating percent live cells at day 7 in LN428 cells transduced with control empty virus vehicle (pSF), virus encoding Spi1 alone, or virus encoding Spi1+IkzF1. All viruses also contain the puromycin-resistance gene. Following transduction, cells were treated with puromycin for 3 days to eliminate un-transduced cells.

FIG. 7B. Dot plots illustrating the percent cells that have phagocytosed pHrodo Red in LN428 cells transduced with control empty virus (EV) or virus encoding Spi1+Ikzf1.

FIG. 7C. Graph illustrating the absolute number of live CD45+ immune-like cells in LN428 cells at day 4, day 7 and day 10 after transduction with control empty virus vehicle (pSF), virus encoding Spi1 alone, or virus encoding Spi1+Ikzf1.

FIG. 8A. Graph illustrating percent live cells at day 7 in LN827 cells transduced with control empty virus vehicle (pSF), virus encoding Spi1 alone, or virus encoding Spi1+IkzF1. All viruses also contain the puromycin-resistance gene. Following transduction, cells were treated with puromycin for 3 days to eliminate un-transduced cells.

FIG. 8B. Graph illustrating percent live cells at day 7 in LN308 cells transduced with control empty virus vehicle (pSF), virus encoding Spi1 alone, or virus encoding Spi1+IkzF1. All viruses also contain the puromycin-resistance gene. Following transduction, cells were treated with puromycin for 3 days to eliminate un-transduced cells.

### DETAILED DESCRIPTION

Various embodiments of the inventions now will be described more fully hereinafter, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements. The term “or” is used herein in both the alternative and conjunctive sense, unless otherwise indicated. The terms “illustrative” and “exemplary” are used to be examples with no indication of quality level.

### A. Definitions

“Transdifferentiation” is the conversion of one cell type, such as a terminally differentiated cell type, into another cell type without the intermediary step of a pluripotent state. Transdifferentiation has been demonstrated in regenerative medicine, such as in the direct conversions of pancreatic exocrine cells into insulin-producing beta cells, and fibroblasts into functional neurons or cardiomyocytes. However, these transdifferentiation efforts were limited to conversions where cell fate determinants had been experimentally identified and validated—a laborious and time intensive process. A barrier to cellular reprogramming lies in the lack of understanding of cell fate determination to direct differentiation, i.e., specific fate determinants expressed temporally and/or deterministically to efficiently transdifferentiate between cell types bypassing the inefficient induced pluripotent stem cells (iPSC) intermediary. Therapeutic applications of transdifferentiation in cancer have been described in very limited cases only when well characterized fate determinants are already known, including leukemic cells converted to cells of other hematologic lineages (e.g., B lymphoma cells into macrophages and invasive breast cancer cells into adipocytes. However, therapeutic reprogramming has not been described in GBM.

An “Antigen-Presenting Cell” (APC) is a cell that displays antigen complexed with major histocompatibility complex II (MHCII) on their surfaces. APCs can process external antigens and present them to other immune cells, such as T cells. Macrophages, B cells and dendritic cells (professional antigen presenting cells) are naturally occurring professional APCs. An APC may also express one or more co-stimulatory molecules.

“Dendritic cells” are antigen-presenting cells having the broadest range of antigen presentation and the ability to activate naïve T cells. Their main function is to process antigen material and present it on the cell surface to T cells. DCs present antigen to both helper and cytotoxic T cells.

“Dendritic-like cells” (also termed “DC-like cells”) are cells that have been transdifferentiated to be able to act as antigen presenting cells. In some embodiments DC-like cells express MHCII and CD11c. In some embodiments, DC-like cells express one or more factors, including but not limited to, CD11c, BDCA-1, CD8, CD8a, CD103, and CD205.

“Macrophages” are a type of white blood cell of the immune system that engulf and digest cellular debris, foreign substances, microbes, cancer cells, etc. in a process called phagocytosis. The engulfed material is then process and antigens are presented at the cell surface in the context of MHCII.

“Macrophage-like cell” are cells that have been transdifferentiated to be able to act as antigen presenting cells. In some embodiments, macrophage-like cells express MHCII and CD11b and/or CD68. In some embodiments, macrophage-like cells express one or more factors including but not limited to, CD14, CD16, CD64, CD71, and CCR5.

“Immune therapy” or “Immunotherapy” is the treatment of disease, such as cancer, by activating or suppressing the immune system. Immunotherapies can be designed to elicit or amplify an immune response.

A “nucleic acid” includes both RNA and DNA. RNA and DNA include, but are not limited to, cDNA, genomic DNA, plasmid DNA, RNA, mRNA, condensed nucleic acid, nucleic acid formulated with cationic lipids, and nucleic acid formulated with peptides or cationic polymers. Nucleic acid also includes modified RNA or DNA.

An “expression vector” refers to a nucleic acid (e.g., RNA or DNA) encoding an expression product (e.g., peptide(s) (i.e., polypeptide(s) or protein(s)) or RNA or microRNA or a small hairpin RNA), such as a transdifferentiation determinant. An expression vector may be, but is not limited to, a virus, a modified virus, a recombinant virus, an attenuated virus, a plasmid, a linear DNA molecule, or an mRNA. An expression vector is capable of expressing one or more polypeptides in a cell, such a mammalian glioblastoma cell. The expression vector may comprise one or more sequences necessary for expression of the encoded expression product. A variety of sequences can be incorporated into an expression vector to alter expression of the coding sequence. The expression vector may comprise one or more of: a 5′ untranslated region (5′ UTR), an enhancer, a promoter, an intron, a 3′ untranslated region (3′ UTR), a terminator, and a polyA signal operably linked to the DNA coding sequence. The nucleic acid encoding the transdifferentiation determinant may be operably linked to a promoter for expressing the transdifferentiation determinant in the GBM cell.

The term “plasmid” refers to a nucleic acid that includes at least one sequence encoding a polypeptide (such as a transdifferentiation determinant) that is capable of being expressed in a glioblastoma cell. A plasmid can be a closed circular DNA molecule. A variety of sequences can be incorporated into a plasmid to alter expression of the coding sequence or to facilitate replication of the plasmid in a cell. Sequences can be used that influence transcription, stability of a messenger RNA (mRNA), RNA processing, or efficiency of translation. Such sequences include, but are not limited to, 5′ untranslated region (5′ UTR), promoter, introns, and 3′ untranslated region (3′ UTR). Plasmids can be manufactured in large scale quantities and/or in high yield. Plasmids can further be manufacture using cGMP manufacturing. Plasmids can be transformed into bacteria, such as E. coli. The DNA plasmids are can be formulated to be safe and effective for injection into a mammalian subject.

A “promoter” is a DNA regulatory region capable of binding an RNA polymerase in a cell (e.g., directly or through other promoter-bound proteins or substances) and initiating transcription of a coding sequence. A promoter may comprise one or more additional regions or elements that influence transcription initiation rate, including, but not limited to, enhancers. A promoter can be, but is not limited to, a constitutively active promoter, a conditional promoter, an inducible promoter, or a cell-type specific promoter. Examples of promoters can be found, for example, in WO 2013/176772. The promoter can be, but is not limited to, CMV promoter, Igκ promoter, mPGK, SV40 promoter, β-actin promoter (such as, but not limited to a human or chicken β-actin promoter), α-actin promoter, SRα promoter, herpes thymidine kinase promoter, herpes simplex virus (HSV) promoter, mouse mammary tumor virus long terminal repeat (LTR) promoter, adenovirus major late promoter (Ad MLP), rous sarcoma virus (RSV) promoter, and EF1α promoter. The CMV promoter can be, but is not limited to, CMV immediate early promoter, human CMV promoter, mouse CNV promoter, and simian CMV promoter. The promoter can also be a hybrid promoter. Hybrid promoters include, but are not limited to, CAG promoter.

“Operably linked” refers to the juxtaposition of two or more components (e.g., a promoter and another sequence element) such that both components function normally and allow the possibility that at least one of the components can mediate a function that is exerted upon at least one of the other components. For example, a promoter operably linked to a coding sequence will direct RNA polymerase mediated transcription of the coding sequence into RNA, including mRNA, which may then be spliced (if it contains introns) and, optionally, translated into a protein encoded by the coding sequence. A coding sequence can be “operably linked” to one or more transcriptional or translational control sequences. A terminator/polyA signal operably linked to a gene terminates transcription of the gene into RNA and directs addition of a poly A signal onto the RNA.

A “heterologous” sequence is a sequence which is not normally present in a cell, genome or gene in the genetic context in which the sequence is currently found. For example, a heterologous sequence can be a coding sequence linked to a different promoter sequence relative to the native coding sequence. A heterologous sequence can differ from its corresponding native sequence in having one or more introns removed. A heterologous sequence can also be present in the context of an expression vector, such as, but not limited to, a plasmid or viral vector.

### B. Transdifferentiation Determinant

A transdifferentiation determinant is a gene which when expressed, or in some cases repressed, either by itself or in combination with one or more additional transdifferentiation determinants, in a differentiated cell, such as a glioblastoma cell, is able to convert (transdifferentiate) the cell into another cell type without the intermediary step of a pluripotent state, such as an antigen presenting cell. In some embodiments, the glioblastoma is a high-grade glioma. In some embodiments, the glioblastoma is a GBM. In some embodiments, the glioblastoma cell is a CSC. Transdifferentiation determinants are provided in the Tables 1-8. A positive regulator of transdifferentiation is a gene whose expression, either by itself or in combination with one or more additional transdifferentiation determinants, in a differentiated cell, such as a glioblastoma cell, is able to convert (transdifferentiate) the cell into another cell type without the intermediary step of a pluripotent state, such as an antigen presenting cell. A negative regulator of transdifferentiation is a gene whose inhibition, either by itself or in combination with one or more additional transdifferentiation determinants, in a differentiated cell, such as a glioblastoma cell, is able to convert (transdifferentiate) the cell into another cell type without the intermediary step of a pluripotent state, such as an antigen presenting cell.

TABLE 1
Human glioblastoma to antigen presenting cell transdifferentiation determinants
(positive and negative regulators or transdifferentiation).

| | NCBI Reference | | NCBI Reference |
|---|---|---|---|
| Gene | Gene Sequence * | Protein | Protein Sequence * |
| AEBP1 | NM_001129.5 | AE binding protein 1 | NP_001120.3 |
| ARID3A | NM_005224.3 | AT-Rich Interaction Domain 3A | NP_005215.1 |
| ATF5 | NM_001193646.2 | activating transcription factor 5 | NP_001180575.1 |
| ATP8B2 | NM_001005855.2 | ATPase phospholipid | NP_001005855.1 |
| | | transporting 8B2 | |
| BASP1 | NM_001271606.2 | Brain Abundant Membrane Attached | NP_001258535.1 |
| | | Signal Protein 1 | |
| BCL11B | NM_022893.4 | BAF chromatin remodeling complex | NP_075044.2 |
| | | subunit BCL11A | |
| BCL6 | NM_001130845.2 | B-cell lymphoma 6 protein | NP_001124317.1 |
| BID | NM_001196.4 | BH3 interacting domain death agonist | NP_001187.1 |
| CBFA2T3 | NM_005187.6 | CBFA2/RUNX1 partner transcriptional | NP_005178.4 |
| | | co-repressor 3 | |
| CIITA | NM_001379332.1 | class II major histocompatibility | NP_001366261.1 |
| | | complex transactivator | |
| CIR1 | NM_004882.4 | corepressor interacting with RBPJ | NP_004873.3 |
| CREG1 | NM_003851.3 | Cellular Repressor Of E1A Stimulated | NP_003842.1 |
| | | Genes 1 | |
| CTSZ | NM_001336.4 | Cathepsin Z | NP_001327.2 |
| CTTN | NM_001184740.2 | cortactin | NP_001171669.1 |
| EGR1 | NM_001964.3 | early growth response 1 | NP_001955.1 |
| ELF4 | NM_001127197.2 | E74 like ETS transcription factor 4 | NP_001120669.1 |
| ETS1 | NM_001143820.2 | ETS proto-oncogene 1, transcription | NP_001137292.1 |
| | | factor | |
| ETV5 | NM_004454.3 | ETS variant transcription factor 5 | NP_004445.1 |
| FOXP1 | NM_001012505.2 | forkhead box P1 | NP_001012523.1 |
| FOXP4 | NM_001012426.2 | forkhead box P4 | NP_001012426.1 |
| GATA3 | NM_001002295.2 | GATA binding protein 3 | NP_001002295.1 |
| GM2A | NM_000405.5 | GM2 ganglioside activator | NP_000396.2 |
| GNS | NM_002076.4 | glucosamine (N-acetyl)-6-sulfatase | NP_002067.1 |
| GTF2IRD1 | NM_016328.3 | GTF2I Repeat Domain Containing 1 | NP_057412.1 |
| HHEX | NM_002729.5 | hematopoietically expressed homeobox | NP_002720.1 |
| HOXB3 | NM_001384747.1 | homeobox B3 | NP_001371676.1 |
| HTATIP2 | NM_001098521.2 | HIV-1 Tat Interactive Protein 2 | NP_001091991.1 |
| IKZF1 | NM_006060.6 | IKAROS family zinc finger 1 | NP_006051.1 |
| IRF5 | NM_001098627.4 | interferon regulatory factor 5 | NP_001092097.2 |
| IRF8 | NM_001363907.1 | interferon regulatory factor 8 | NP_001350836.1 |
| KIF21A | NM_001173464.2 | kinesin family member 21A | NP_001166935.1 |
| KLF4 | NM_001314052.2 | Kruppel like factor 4 | NP_001300981.1 |
| KLF11 | NM_003597.5 | Kruppel like factor 11 | NP_003588.1 |
| KLF12 | NM_007249.5 | Kruppel like factor 12 | NP_009180.3 |
| KMT2E | NM_018682.4 | lysine methyltransferase 2E | NP_061152.3 |
| LDB1 | NM_001113407.3 | LIM domain binding 1 | NP_001106878.1 |
| LEF1 | NM_001130713.3 | Lymphoid enhancer-binding factor 1 | NP_001124185.1 |
| LMO2 | NM_005574.4 | LIM Domain Only 2 | NP_005565.2 |
| LOXL2 | NM_002318.3 | lysyl oxidase like 2 | NP_002309.1 |
| MAGED1 | NM_001005333.2 | MAGE family member D1 | NP_001005333.1 |
| MMP14 | NM_004995.4 | matrix metallopeptidase 14 | NP_004986.1 |
| MREG | NM_001372188.1 | melanoregulin | NP_001359117.1 |
| MXD1 | NM_002357.4 | MAX dimerization protein 1 | NP_002348.1 |
| MYBL1 | NM_001080416.4 | MYB proto-oncogene like 1 | NP_001073 885.1 |
| NAB2 | NM_005967.4 | NGFI-A binding protein 2 | NP_005958.1 |
| NCOA3 | NM_181659.3 | nuclear receptor coactivator 3 | NP_858045.1 |
| NFATC2 | NM_001136021.3 | nuclear factor of activated T cells 2 | NP_001129493.1 |
| NFE2L1 | NM_003204.3 | nuclear factor, erythroid 2 like 1 | NP_003195.1 |
| NOTCH2 | NM_024408.4 | notch receptor 2 | NP_077719.2 |
| NR1H3 | NM_005693.4 | nuclear receptor subfamily 1 group H | NP_005684.2 |
| | | member 3 | |
| PAWR | NM_001354732.2 | pro-apoptotic WT1 regulator | NP_001341661.1 |
| PCGF2 | NM_001369614.1 | polycomb group ring finger 2 | NP_001356543.1 |
| PDLIM1 | NM_020992.4 | PDZ and LIM domain 1 | NP_066272.1 |
| PLAGL1 | NM_001080955.3 | PLAG1 like zinc finger 1 | NP_001074424.1 |
| PLCG1 | NM_002660.3 | phospholipase C gamma 1 | NP_002651.2 |
| POU6F1 | NM_001330422.2 | POU class 6 homeobox 1 | NP_001317351.1 |
| PRKCB | NM_002738.7 | Protein kinase C Beta | NP_002729.2 |
| PTPN14 | NM_005401.5 | protein tyrosine phosphatase non- | NP_005392.2 |
| | | receptor type 14 | |
| RBI | NM_000321.3 | RB Transcriptional Corepressor 1 | NP_000312.2 |
| RBFOX2 | NM_001031695.4 | RNA binding fox-1 homolog 2 | NP_001026865.1 |
| RBPMS | NM_001008710.3 | RNA binding protein, mRNA | NP_001008710.1 |
| | | processing factor | |
| RORA | NM_134261.3 | RAR related orphan receptor A | NP_599023.1 |
| RUNX3 | NM_001031680.2 | RUNX family transcription factor 3 | NP_001026850.1 |
| SALL2 | NM_001291446.2 | Spalt-like transcription factor 2 | NP_001278375.1 |
| SATB1 | NM_001131010.4 | Special AT-rich sequence-binding | NP_001124482.1 |
| | | protein-1 | |
| SOX13 | NM_005686.3 | SRY-box transcription factor 13 | NP_005677.2 |
| SPI1 | NM_001080547.2 | PU.1 | NP_001074016.1 |
| STAT6 | NM_001178078.2 | signal transducer and activator of | NP_001171549.1 |
| | | transcription 6 | |
| TCF7 | NM_003202.5 | Transcription factor 7 | NP_003193.2 |
| TCF19 | NM_001077511.2 | transcription factor 19 | NP_001070979.1 |
| TDP2 | NM_016614.3 | tyrosyl-DNA phosphodiesterase 2 | NP_057698.2 |
| TFEB | NM_001271944.2 | transcription factor EB | NP_001258873.1 |
| TFEC | NM_012252.4 | Transcription factor EC | NP_036384.1 |
| TLE2 | NM_003260.5 | TLE family member 2, transcriptional | NP_003251.2 |
| | | corepressor | |
| TSHZ2 | NM_173485.6 | teashirt zinc finger homeobox 2 | NP_775756.3 |
| USF1 | NM_001276373.2 | upstream transcription factor 1 | NP_001263302.1 |
| USF2 | NM_003367.4 | upstream transcription factor 2, c-fos | NP_003358.1 |
| | | interacting | |
| ZBTB34 | NM_001099270.2 | Zinc Finger And BTB Domain | NP_001092740.1 |
| | | Containing 34 | |
| ZEB1 | NM_001128128.3 | zinc finger E-box binding homeobox 1 | NP_001121600.1 |
| ZFP91 | NM_053023.5 | ZFP91 zinc finger protein, atypical E3 | NP_444251.1 |
| | | ubiquitin ligase | |
| ZNF74 | NM_001256523.2 | Zinc Finger Protein 74 | NP_001243452.1 |
| ZNF280B | NM_080764.4 | zinc finger protein 280B | NP_542942.2 |
| ZNF366 | NM_152625.3 | Zinc finger protein 366 (Dendritic Cell- | NP_689838.1 |
| | | Specific Transcript Protein) | |
| ZNF483 | NM_133464.5 | zinc finger protein 483 | NP_597721.2 |
| ZNF507 | NM_001136156.2 | zinc finger protein 507 | NP_001129628.1 |
| ZNF827 | NM_001306215.2 | zinc finger protein 827 | NP_001293144.1 |

* For some genes and proteins, multiple isoforms are known. The indicated NCBI Reference number represents a single isoform and is provided as an exemplary sequence. Use of any of the known isoforms is contemplated.

TABLE 2
Mouse glioblastoma to antigen presenting cell transdifferentiation determinants.

| | NCBI Reference | | NCBI Reference |
|---|---|---|---|
| Gene | Gene Sequence * | Protein | Protein Sequence * |
| AES | NM_001276288.1 | Amino Enhancer Of Split | NP_001121.2 |
| ARF2 | NM_001304574.1 | ADP-ribosylation factor 2 | NP_001291503.1 |
| ATOX1 | NM_009720.3 | antioxidant 1 copper chaperone | NP_033850.1 |
| BATF3 | NM_030060.2 | basic leucine zipper transcriptional | NP_084336.1 |
| | | factor ATF-like 3 | |
| BCL11A | NM_001159289.1 | B cell CLL/lymphoma 11A (zinc finger | NP_001152761.1 |
| | | protein | |
| CBFA2T3 | NM_001109873.1 | CBFA2/RUNX1 translocation partner | NP_001103343.1 |
| CEBPA | NM_001287514.1 | CCAAT/enhancer binding protein | NP_001274443.1 |
| | | (C/EBP), alpha | |
| CEBPB | NM_001287738.1 | CCAAT/enhancer binding protein | NP_001274667.1 |
| | | (C/EBP), beta | |
| PPARD | NM_011145.3 | peroxisome proliferator activator | NP_035275.1 |
| | | receptor delta | |
| ETV3 | NM_001083318.2 | ets variant 3 | NP_001076787.1 |
| ETV6 | NM_001303102.1 | ets variant 6 | NP_001290031.1 |
| GLMP | NM_020003.1 | glycosylated lysosomal membrane protein | NP_064387.1 |
| HELZ2 | NM_183162.2 | helicase with zinc finger 2, | NP_898985.2 |
| | | transcriptional coactivator | |
| HHEX | NM_008245.3 | hematopoietically expressed homeobox | NP_032271.1 |
| ID2 | NM_010496.3 | inhibitor of DNA binding 2 | NP_034626.1 |
| IKZF3 | NM_011771.1 | IKAROS family zinc finger 3 | NP_035901.1 |
| IRF5 | NM_001252382.1 | interferon regulatory factor 5 | NP_001239311.1 |
| IRF8 | NM_001301811.1 | interferon regulatory factor 8 | NP_001288740.1 |
| LMO2 | NM_001142335.1 | LIM domain only 2 | NP_001135807.1 |
| MAFB | NM_010658.3 | v-maf musculoaponeurotic fibrosarcoma | NP_034788.1 |
| | | oncogene family, protein B | |
| MAZ | NM_001372521.1 | MYC-associated zinc finger protein | NP_001359450.1 |
| | | (purine-binding transcription factor) | |
| MXD1 | NM_010751.3 | MAX dimerization protein 1 | NP_034881.2 |
| MYCL | NM_001303121.1 | v-myc avian myelocytomatosis viral | NP_001290050.1 |
| | | oncogene lung carcinoma derived | |
| POU2AF1 | NM_011136.2 | POU domain, class 2, associating | NP_035266.1 |
| | | factor 1 | |
| SPI1 | NM_001378898.1 | PU.1 | NP_001365827.1 |
| SPIB | NM_019866.1 | Spi-B transcription factor (Spi-1/PU.1 | NP_063919.1 |
| | | related) | |
| TAF10 | NM_020024.3 | TATA-box binding protein associated | NP_064408.2 |
| | | factor 10 | |
| TFE3 | NM_001105196.1 | transcription factor E3 | NP_001098666.1 |
| VAV1 | NM_001163815.1 | vav 1 oncogene | NP_001157287.1 |
| ZFP384 | NM_001252083.1 | zinc finger protein 384 | NP_001239012.1 |

* For some genes and proteins, multiple isoforms are known. The indicated NCBI Reference number represents a single isoform and is provided as an exemplary sequence. Use of any of the known isoforms is contemplated.

In some embodiments, a transdifferentiation determinant comprises any of the genes or proteins listed in Tables 1-8. In some embodiments, increasing expression of a transdifferentiation determinant comprises expressing in the glioblastoma cell a nucleic acid comprising the coding sequence of any of the genes of Tables 1-7, or a functional equivalent thereof, a nucleic acid encoding any of the proteins of Tables 1-7, or a nucleic acid encoding a protein having the activity of any of the proteins of Tables 1-7. In some embodiments, a decreasing expression of a transdifferentiation determinant comprises administering to a glioblastoma cell an expression inhibitor that inhibits expression of any of the transdifferentiation determinants of Table 8.

In some embodiments, one, two, three, four, or more nucleic acids that increase and/or decrease expression of one or more glioblastoma to antigen presenting cell transdifferentiation determinants are delivered to a glioblastoma or to glioblastoma cells to transdifferentiate glioblastoma cells to antigen presenting cells. In some embodiments, the glioblastoma cell is a high-grade glioma. In some embodiments, the glioblastoma cell is a GBM cell. In some embodiments, a first nucleic acid is delivered to a glioblastoma cell. In some embodiments a first nucleic acid and a second nucleic acid are delivered to a glioblastoma cell. In some embodiments a first nucleic acid, a second nucleic acid, and a third nucleic acid are delivered to a glioblastoma cell. In some embodiments a first, a second, a third, and a fourth transdifferentiation determinant are delivered to a glioblastoma cell.

In some embodiments, the glioblastoma or glioblastoma cell is a human glioblastoma or human glioblastoma cell, and the transdifferentiation determinant is a human gene. In some embodiments, a human transdifferentiation determinant is selected from the group consisting of: SPI1, IKZF1, ARID3A, ATF5, BASP1, BCL6, CIITA, CIR1, CREG1, CTSZ, ELF4, FOXP1, GNS, HHEX, HTATIP2, IRF5, KLF11, KMT2E, LDB1, LMO2, MMP14, MREG, MXD1, NAB2, NCOA3, NFE2L1, NOTCH2, NR1H3, PRKCB, SATB1, STAT6, TDP2, TFEC, USF1, USF2, ZBTB34, ZFP91, and ZNF366. In some embodiments, increasing and/or decreasing expression of one or more transdifferentiation determinants can be used to transdifferentiate a glioblastoma cancer stem cell (CSC) to an antigen presenting cell. In some embodiments, the increasing and/or decreasing expression of one or more transdifferentiation determinants can be used to transdifferentiate a glioblastoma cell to an antigen presenting cell. The antigen presenting cell can be, e.g., a dendritic-like cell or a macrophage-like cell. In some embodiments, the induce antigen presenting cell is able to stimulate naïve T cells.

In some embodiments, increasing and/or decreasing expression of one or more transdifferentiation determinants is used to transdifferentiate a glioblastoma cell to a dendritic-like cell. In some embodiments, the glioblastoma or glioblastoma cell is a mouse glioblastoma or mouse glioblastoma cell, and the transdifferentiation determinant is a mouse gene. In some embodiments, the mouse transdifferentiation determinant is selected from the group consisting of: IRF5, CBFA2T3, IRF8, ATOX1, SPI1, BCL11A, ID2, MYCL, BATF3, HHEX, SPIB, VAV1, ETV6, MXD1, ETV3, LMO2, AES, GLMP, CEBPA, and MAZ. In some embodiments, the glioblastoma or glioblastoma cell is a human glioblastoma cancer stem cell or human glioblastoma cell, and the transdifferentiation determinant is a human gene. In some embodiments, the human transdifferentiation determinant is selected from the group consisting of: SPI1, IKZF1, FOXP1, CTSZ, HHEX, TFEC, HTATIP2, KLF11, PRKCB, USF1, TDP2, BCL6, CREG1, ZNF366, LDB1, KMT2E, CIITA, ZFP91, ELF4, NCOA3, ZBTB34, ARID3A, SATB1, STAT6, LMO2, and NAB2.

In some embodiments, increasing and/or decreasing expression of one or more transdifferentiation determinants is used to transdifferentiate a glioblastoma cell to a macrophage-like cell. In some embodiments, the glioblastoma cell is a GBM cell. In some embodiments, the GBM or GBM cell is a human GBM cancer stem cell or a human GBM cell, and the transdifferentiation determinant is a human gene. In some embodiments, the human transdifferentiation determinant is selected from the group consisting of: SPI1, IKZF1, CTSZ, TFEC, HTATIP2, FOXP1, CREG1, TDP2, PRKCB, CIR1, NR1H3, KLF11, GNS, MMP14, HHEX, BASP1, KMT2E, ATF5, NFE2L1, IRF5, SATB1, ARID3A, ZBTB34, NOTCH2, MXD1, USF2, and MREG.

In some embodiments, the glioblastoma or glioblastoma cell is a mouse glioblastoma or mouse glioblastoma cell, and the transdifferentiation determinant is a mouse gene. In some embodiments, the mouse transdifferentiation determinant is selected from the group consisting of: SPI1, IRF5, IRF8, TFE3, CEBPA, BCL11A, CEBPB, SPIB, POU2AF1, HELZ2, IKZF3, MAFB, LMO2, VAV1, ARF2, CBFA2T3, MAZ, PPARD, TAF10, and ZFP384.

In some embodiments, at least two different nucleic acids for increasing or decreasing expression of one or more transdifferentiation determinants are administered to the glioblastoma or glioblastoma cells. The least two different transdifferentiation determinants are independently selected from any of the transdifferentiation determinants listed in Tables 1-8. In some embodiments, at least one of the at least two different transdifferentiation determinants comprises SPI1 or IKZF1. In some embodiments, at least one of the at least two different transdifferentiation determinants comprises SPI1. In some embodiments, at least one of the at least two different transdifferentiation determinants comprises IKZF1. In some embodiments, the at least two different transdifferentiation determinants comprise SPI1 and IKZF1. In some embodiments, the at least two different transdifferentiation determinant further comprises a third transdifferentiation determinant. The third transdifferentiation determinant can be selected from the group consisting of: ARID3A, ATF5, BASP1, BCL6, CIITA, CIR1, CREG1, CTSZ, ELF4, FOXP1, GNS, HHEX, HTATIP2, IRF5, KLF11, KMT2E, LDB1, LMO2, MMP14, MREG, MXD1, NAB2, NCOA3, NFE2L1, NOTCH2, NR1H3, PRKCB, SATB1, STAT6, TDP2, TFEC, USF1, USF2, ZBTB34, ZFP91, ZNF366. In some embodiments, the three transdifferentiation determinants include SP1, IKZF1, and a third transdifferentiation determinant selected from the group consisting of: CTSZ, HTATIP2, TFEC, PRKCB, TDP2, CREG1, FOXP1, HHEX, and KMT2E. In some embodiments, the three transdifferentiation determinants include SP1, IKZF1, and a third transdifferentiation determinant selected from the group consisting of: CTSZ, HTATIP2, TFEC, PRKCB, TDP2, and CREG1.

In some embodiments, at least one of the at least two different transdifferentiation determinant comprises ID2.

In some embodiments, at least three different nucleic acids for increasing and/or decreasing expression at least three different transdifferentiation determinants are administered to the glioblastoma or glioblastoma cells. The at least three different transdifferentiation determinants are independently selected from any of the transdifferentiation determinants listed in Tables 1-8. In some embodiments, at least one of the at least three different transdifferentiation determinants comprises SPI1 or IKZF1. In some embodiments, at least one of the at least three different transdifferentiation determinants comprises SPI1. In some embodiments, at least one of the at least three different transdifferentiation determinants comprises IKZF1. In some embodiments, at least two of the at least three different transdifferentiation determinants comprise SPI1 and IKZF1.

In some embodiments, the at least three transdifferentiation determinants comprise a first transdifferentiation determinant, a second transdifferentiation determinant, and a third transdifferentiation determinant, wherein the first transdifferentiation determinant comprises SPI1, the second transdifferentiation determinant comprises IKZF1, and the third transdifferentiation determinant is selected from the group consisting of: AES, ARF2, ARID3A, ATOX1, BASP1, BATF3, BCL11A, BCL11B, BCL6, CBFA2T3, CEBPA, CEBPB, CREG1, CTSZ, PPARD, ETV3, ETV6, GATA3, GLMP, GTRF2IRD1, HELZ2, HHEX, HTATIP2, ID2, IKZF3, IRF5, IRF8, KLF11, LEF1, LMO2, MAFB, MAZ, MXD1, MYCL, POU2AF1, PRKCB, RB1, RBFOX2, SALL2, SATB1, SPIB, TAF10, TCF7, TFE3, TFEC, USF1, VAV1, ZBTB34, ZFP384, ZNF366, and ZNF74. In some embodiments, the third transdifferentiation determinant is selected from the group consisting of: IRF8, ATOX1, BCL11A, ID2, BATF3, and IKZF3. In some embodiments, the at least three transdifferentiation determinants comprises a fourth transdifferentiation determinant selected from the group consisting of: AES, ARF2, ARID3A, ATOX1, BASP1, BATF3, BCL11A, BCL11B, BCL6, CBFA2T3, CEBPA, CEBPB, CREG1, CTSZ, PPARD, ETV3, ETV6, GATA3, GLMP, GTRF2IRD1, HELZ2, HHEX, HTATIP2, ID2, IKZF3, IRF5, IRF8, KLF11, LEF1, LMO2, MAFB, MAZ, MXD1, MYCL, POU2AF1, PRKCB, RB1, RBFOX2, SALL2, SATB1, SPIB, TAF10, TCF7, TFE3, TFEC, USF1, VAV1, ZBTB34, ZFP384, ZNF366, and ZNF74. In some embodiments, the fourth different transdifferentiation determinant comprises ID2.

In some embodiments, the first transdifferentiation determinant comprises SPI1, the second transdifferentiation determinant comprises IRF8, and the third transdifferentiation determinant comprises BATF3. In some embodiments, the at least three different transdifferentiation determinant further comprises a fourth transdifferentiation determinant. In some embodiments, the fourth transdifferentiation determinant comprises ID2.

In some embodiments, the first transdifferentiation determinant comprises SPI1, the second transdifferentiation determinant comprises ID2, and the third transdifferentiation determinant comprises ATOX1. In some embodiments, the combination of SPI1, ID2, and ATOX1 further comprises a fourth transdifferentiation determinant comprising BCL11A.

For administration in vivo, any of the described nucleic acids for increasing and/or decreasing expression the transdifferentiation determinants may be combined with one or more pharmaceutically acceptable excipients. Pharmaceutically acceptable excipients (“excipients”) are substances other than the Active Pharmaceutical ingredient (API, therapeutic product; e.g., nucleic acid encoding a transdifferentiation determinant) that are intentionally included in the drug delivery system. Excipients do not exert or are not intended to exert a therapeutic effect at the intended dosage. Excipients may act to a) aid in processing of the drug delivery system during manufacture, b) protect, support or enhance stability, bioavailability or patient acceptability of the API, c) assist in product identification, and/or d) enhance any other attribute of the overall safety, effectiveness, or delivery of the API during storage or use. A pharmaceutically acceptable excipient may or may not be an inert substance. Excipients include, but are not limited to: agents that enhance transfection, absorption enhancers, anti-adherents, anti-foaming agents, anti-oxidants, binders, buffering agents, carriers, coating agents, colors, delivery enhancers, delivery polymers, dextran, dextrose, diluents, disintegrants, emulsifiers, extenders, fillers, flavors, glidants, humectants, lubricants, oils, polymers, preservatives, saline, salts, solvents, sugars, suspending agents, sustained release matrices, thickening agents, tonicity agents, vehicles, water-repelling agents, and wetting agents. Agents that enhance transfection include, but are not limited to, lipids, cationic lipids, lipids, polycations, cell-penetrating peptides, and combinations thereof.

### C. Administration of a Transdifferentiation Determinant to a Glioblastoma Cell

The described compositions and methods can be used to transdifferentiate GBM cells into antigen presenting cells capable of presenting tumor antigens to the immune system. The antigen presenting cells gain functional characteristics of dendritic cells or macrophages. Described are compositions and methods for generating antigen presenting cells in situ directly from glioblastoma cells. The locally created antigen presenting cells are located in the tumor microenvironment (TME) with access to tumor neoantigens.

The described nucleic acids for increasing and/or decreasing expression transdifferentiation determinants can be delivered to glioblastoma cells using methods known in the art. In some embodiments, the nucleic acids are administered to a subject to the subject. In some embodiments, the nucleic acids are administered to a glioblastoma in the subject.

A nucleic acid encoding a transdifferentiation determinant can be a DNA or RNA. The DNA or RNA can be single or double stranded, linear or circular, relaxed or supercoiled. The nucleic acid can be an expression vector, a plasmid or a viral nucleic acid (i.e., part of a viral vector). An RNA can be an mRNA or a microRNA or a small hairpin RNA.

In some embodiments, the expression inhibitor in a nucleic acid-based expression inhibitor. A nucleic acid-based expression inhibitor can be, but is not limited to, an RNA interfering agent, such as an siRNA shRNA, or miRNA, an antisense oligonucleotide, or a gene for expressing an RNA interfering agent or antisense oligonucleotide.

In some embodiments, a nucleic acid encoding a transdifferentiation determinant is administered to a glioblastoma in vivo. The nucleic acid is administered to the glioblastoma such that the nucleic acid is delivered to one or more glioblastoma cells in the glioblastoma and expressed in the glioblastoma cells.

In some embodiments an expression inhibitor is administered to a glioblastoma cell in vivo. The expression inhibitor is administered to the glioblastoma such that the expression inhibitor is delivered to one or more glioblastoma cells in the glioblastoma and inhibits expression of a transdifferentiation determinant in the glioblastoma cells.

In some embodiments, the nucleic acid is administered to the glioblastoma via a non-viral vector. Non-viral methods of delivery of nucleic acid to cells in vivo include, but are not limited to, direct injection (with or without electroporation), needless injection (with or without electroporation), microprojectile bombardment (e.g., gene gun), hydrodynamic injection, magneto-fection, sono-poration (e.g., ultrasound-mediated delivery), photo-poration, and hydro-poration. The nucleic acid sequence can be in a nanoparticle, a lipid nanoparticle, a liposome, a lipoplex, a polyplex, a lipopolyplex, or other non-viral particle or complex. In some embodiments, the nucleic acid is administered to the glioblastoma via a viral vector. A viral vector may be administered to cells in vivo by methods know in the art, included, but not limited to direct injection into a tumor and intravascular injection. The viral vector can be, but is not limited to, an adenovirus, and adeno-associated virus, a retrovirus, or an alphavirus. A retrovirus can be, but is not limited to, a lentivirus. An alphavirus can be, but is not limited to a Semliki Forest virus.

The nucleic acid(s) encoding a transdifferentiation determinant(s) or expression inhibitor may be administered as a single dose or as multiple doses. Multiple doses include multiple doses of the same transdifferentiation determinant(s), multiple doses of different transdifferentiation determinant(s), or combinations thereof. Administration of multiple doses includes, for example two doses, three doses, four doses, five doses, six doses, or more. The multiple doses can be administered to a subject over days, weeks or months. For administration of two of more transdifferentiation determinants, the transdifferentiation determinants may be administered simultaneously (or on the same day) or sequentially (on different days). For sequential administration, the two different transdifferentiation determinants may be administered to the glioblastoma up to 30 days or more apart.

In some embodiments, methods of treating glioblastoma are described. The methods comprise administering one or more nucleic acids encoding one or more transdifferentiation determinants to glioblastoma cells in a subject in vivo, wherein the one or more transdifferentiation determinants are expressed. In some embodiments, the methods comprise administering one or more expression inhibitors that inhibit expression one or more of the described negative regulators of transdifferentiation. In some embodiments, the methods comprise administering one or more nucleic acids encoding one or more transdifferentiation determinants and one or more expression inhibitors that inhibit expression one or more negative regulators to glioblastoma cells in a subject in vivo. Expression of the one or more transdifferentiation determinants and/or inhibition of one or more negative determinants in glioblastoma cells in the subject leads to transdifferentiation of glioblastoma cell to antigen presenting cells.

In some embodiments, methods of eliciting an immune response against a glioblastoma are described. The methods comprise administering one or more nucleic acids encoding one or more transdifferentiation determinants to glioblastoma cells in a subject in vivo, wherein the one or more transdifferentiation determinants are expressed. In some embodiments, the eliciting an immune response comprises administering one or more expression inhibitors that inhibit expression one or more of the described negative regulators of transdifferentiation. In some embodiments, the eliciting an immune response comprises administering one or more nucleic acids encoding one or more transdifferentiation determinants and one or more expression inhibitors that inhibit expression one or more negative regulators of transdifferentiation to glioblastoma cells in a subject in vivo. Expression of the one or more transdifferentiation determinants and/or inhibition of one or more negative determinants in glioblastoma cells in the subject leads to transdifferentiation of glioblastoma cell to antigen presenting cells.

Expression of the transdifferentiation determinants and/or inhibition of one or more negative determinants in glioblastoma cells transdifferentiate the glioblastoma cells into antigen presenting cells. Transdifferentiation of glioblastoma cells into antigen presenting cells can result in stimulation of the immune cells to attack the glioblastoma. In some embodiments, conversion of as little as 1% of the cells in the glioblastoma tumor is sufficient to induce an immune response against the tumor. In some embodiments, using the described methods or nucleic acids, at least 1%, at least 2%, at least 3%, at least 4%, or at least 5% of treated glioblastoma cells are transdifferentiated. In some embodiments, using the described methods, 1-5%, 1-10%, 1-15%, 1-20%, 1-25% or more of treated glioblastoma cells are transdifferentiated. In some embodiments, using the described methods, at least 6%, at least 7%, at least 8%, at least 9%, at least 10%, at least 15%, or at least 20% of the treated glioblastoma cells are transdifferentiated.

In some embodiments, the expression of the transdifferentiation determinants reduces growth of the glioblastoma.

### D. Listing of Embodiments

1. A method of treating glioblastoma comprising: administering to the glioblastoma in a subject one or more nucleic acids that increase and/or decrease expression of one or more glioblastoma to antigen presenting cell transdifferentiation determinants thereby increasing and/or decreasing expression of one or more of the transdifferentiation determinants in glioblastoma cells of the glioblastoma.

2. The method of embodiment 1, wherein the glioblastoma is a high-grade glioma or a glioblastoma multiforme.

3. The method of embodiment 1 or 2, wherein increasing and/or decreasing expression of the one or more transdifferentiation determinants in the glioblastoma cells results in transdifferentiation of one or more of the glioblastoma cells into antigen presenting cells.

4. The method of embodiment 3, wherein the antigen presenting cells are dendritic cell-like and/or macrophage-like.

5. The method of embodiment 1 or 2, wherein increasing and/or decreasing expression of the one or more transdifferentiation determinants in the glioblastoma cells results in reduced growth rate of the glioblastoma cells.

6. The method of any one of embodiments 1-5, wherein the one or more transdifferentiation determinants are selected from the transdifferentiation determinants in Tables 1-8.

7. The method of embodiment 6, wherein the one or more transdifferentiation determinants are selected from the list consisting of: SPI1, IKZF1, ARID3A, ATF5, BASP1, BCL6, CIITA, CIR1, CREG1, CTSZ, ELF4, FOXP1, GNS, HHEX, HTATIP2, IRF5, KLF11, KMT2E, LDB1, LMO2, MMP14, MREG, MXD1, NAB2, NCOA3, NFE2L1, NOTCH2, NR1H3, PRKCB, SATB1, STAT6, TDP2, TFEC, USF1, USF2, ZBTB34, ZFP91, ZNF366.

8. The method of embodiment 6, wherein the one or more transdifferentiation determinants are selected from the list consisting of: SPI1, IKZF1, GATA3, BATF3, IRF8, ID2, IRF5, CBFA2T3, ATOX1, BCL11A, and MYCL.

9. The method of embodiment 6, wherein the one or more transdifferentiation determinants are selected from the list consisting of: IRF5, CBFA2T3, IRF8, ATOX1, SPI1, BCL11A, ID2, MYCL, BATF3, HHEX, SPIB, VAV1, ETV6. MXD1, ETV3, LMO2, AES, GLMP, CEBPA, and MAZ.

10. The method of embodiment 6, wherein the one or more transdifferentiation determinants are selected from the list consisting of: SPI1, IRF5, IRF8, TFE3, CEBPA, BCL11A, CEBPB, SPIB, POU2AF1, HELZ2, IKZF3, MAFB, LMO2, VAV1, ARF2, CBFA2T3, MAZ, PPARD, TAF10, and ZFP384.

11. The method of embodiment 6 wherein the one or more transdifferentiation determinants are selected from the list consisting of: IRF5, CBFA2T3, IRF8, ATOX1, SPI1, BCL11A, ID2, MYCL, and BATF3.

12. The method of embodiment 6, wherein the glioblastoma is a human glioblastoma and the one or more transdifferentiation determinants are selected from Tables 5-8.

13. The method of embodiment 12, wherein the glioblastoma is a human glioblastoma and the one or more transdifferentiation determinants are selected from the list consisting of: SPI1, IKZF1, FOXP1, CTSZ, HHEX, TFEC, HTATIP2, KLF11, PRKCB, USF1, TDP2, BCL6, CREG1, ZNF366, LDB1, KMT2E, CIITA, ZFP91, ELF4, NCOA3, FOXP1, CIR1, NR1H3, GNS, MMP14, BASP1, ATF5, NFE2L1, and IRF5.

14. The method of embodiment 12, wherein the glioblastoma is a human glioblastoma and the one or more transdifferentiation determinants are selected from the list consisting of: SPI1, IKZF1, CTSZ, PRKCB, HTATIP2, TFEC, USF1, ZBTB34, TDP2, ARID3A, SATB1, FOXP1, STAT6, CREG1, LMO2, ZNF366, HHEX, CIITA, KMT2E, NAB2, MMP14, NR1H3, CIR1, NFE2L1, GNS, NOTCH2, MXD1, USF2, and MREG.

15. The method of any one of embodiments 1-14, wherein the method comprises administering to the glioblastoma two or more nucleic acids that increase and/or decrease expression of two or more transdifferentiation determinants.

16. The method of embodiment 15, wherein two or more transdifferentiation determinants are selected from the group consisting of: IRF8, ATOX1, SPI1, BCL11A, ID2, BATF3, IKZF1, IKZF3 and GATA3.

17. The method of embodiment 16 wherein the two or more transdifferentiation determinants comprise SPI1 and IKZF1.

18. The method of embodiment 17, wherein the method further comprises administering a nucleic acid encoding one or more of ID2, IRF8, BATF3, ATOX1, and BCL11A.

19. The method of any one of embodiments 1-18, wherein the method comprises administering to the glioblastoma three or more nucleic acids that increase and/or decrease expression of three or more different transdifferentiation determinants.

20. The method of embodiment 19, wherein the three or more transdifferentiation determinants are selected from the group consisting of: SPI1, IKZF1, IRF8, ATOX1, BCL11A, ID2, BATF3, IKZF3, and GATA3.

21. The method of embodiment 20, wherein the three or more transdifferentiation determinants comprise at least SPI1 and IKZF1.

21.1. The method of any one of embodiments 1-18, wherein the method comprises administering to the glioblastoma four or more nucleic acids that increase and/or decrease expression of four or more different transdifferentiation determinants.

22.2. The method of any one of embodiments 1-18, wherein the method comprises administering to the glioblastoma five nucleic acids that increase and/or decrease expression of five different transdifferentiation determinants.

22.3 The method of embodiment 21.1 or 21.2, wherein at least two of the transdifferentiation determinants are selected from the group consisting of: SPI1, IKZF1, CTSZ, USF1, and PRKCB.

22. A method for transdifferentiation of glioblastoma cells into antigen presenting cells comprising: administering to the glioblastoma one or more nucleic acids that increase and/or decrease expression of one or more glioblastoma to antigen presenting cell transdifferentiation determinants thereby increasing and/or decreasing expression of one or more of the transdifferentiation determinants in the glioblastoma cells.

23. The method of embodiment 22, wherein the glioblastoma cell is a high-grade glioma cell or a glioblastoma multiforme cell.

24. The method of embodiment 22 or 23, wherein the antigen presenting cells are dendritic cell-like or macrophage-like.

25. The method of any one of embodiments 22-24, wherein the one or more transdifferentiation determinants are selected from the transdifferentiation determinants in Tables 1-8.

26. The method of embodiment 25, wherein the one or more transdifferentiation determinants are selected from the list consisting of: SPI1, IKZF1, ARID3A, ATF5, BASP1, BCL6, CIITA, CIR1, CREG1, CTSZ, ELF4, FOXP1, GNS, HHEX, HTATIP2, IRF5, KLF11, KMT2E, LDB1, LMO2, MMP14, MREG, MXD1, NAB2, NCOA3, NFE2L1, NOTCH2, NR1H3, PRKCB, SATB1, STAT6, TDP2, TFEC, USF1, USF2, ZBTB34, ZFP91, ZNF366.

27. The method of embodiment 25, wherein the one or more transdifferentiation determinants are selected from the list consisting of: SPI1, IKZF1, GATA3, BATF3, IRF8, ID2, IRF5, CBFA2T3, ATOX1, BCL11A, and MYCL.

28. The method of embodiment 25, wherein the one or more transdifferentiation determinants are selected from the list consisting of: IRF5, CBFA2T3, IRF8, ATOX1, SPI1, BCL11A, ID2, MYCL, BATF3, HHEX, SPIB, VAV1, ETV6. MXD1, ETV3, LMO2, AES, GLMP, CEBPA, and MAZ.

29. The method of embodiment 25, wherein the one or more transdifferentiation determinants are selected from the list consisting of: SPI1, IRF5, IRF8, TFE3, CEBPA, BCL11A, CEBPB, SPIB, POU2AF1, HELZ2, IKZF3, MAFB, LMO2, VAV1, ARF2, CBFA2T3, MAZ, PPARD, TAF10, and ZFP384.

30. The method of embodiment 25, wherein the one or more transdifferentiation determinants are selected from the list consisting of: IRF5, CBFA2T3, IRF8, ATOX1, SPI1, BCL11A, ID2, MYCL, and BATF3.

31. The method of embodiment 25, wherein the glioblastoma is a human glioblastoma and the one or more transdifferentiation determinants are selected from Tables 5-8.

32. The method of embodiment 31, wherein the glioblastoma is a human glioblastoma and the one or more transdifferentiation determinants are selected from the list consisting of: SPI1, IKZF1, FOXP1, CTSZ, HHEX, TFEC, HTATIP2, KLF11, PRKCB, USF1, TDP2, BCL6, CREG1, ZNF366, LDB1, KMT2E, CIITA, ZFP91, ELF4, NCOA3, FOXP1, CIR1, NR1H3, GNS, MMP14, BASP1, ATF5, NFE2L1, and IRF5.

33. The method of embodiment 31, wherein the glioblastoma is a human glioblastoma and the one or more transdifferentiation determinants are selected from the list consisting of: SPI1, IKZF1, CTSZ, PRKCB, HTATIP2, TFEC, USF1, ZBTB34, TDP2, ARID3A, SATB1, FOXP1, STAT6, CREG1, LMO2, ZNF366, HHEX, CIITA, KMT2E, NAB2, MMP14, NR1H3, CIR1, NFE2L1, GNS, NOTCH2, MXD1, USF2, and MREG.

34. The method of any one of embodiments 22-33, wherein the method comprises administering to the glioblastoma two or more nucleic acids that increase and/or decrease expression of two or more transdifferentiation determinants.

35. The method of embodiment 34, wherein two or more transdifferentiation determinants are selected from the group consisting of: IRF8, ATOX1, SPI1, BCL11A, ID2, BATF3, IKZF1, IKZF3 and GATA3.

36. The method of embodiment 35, wherein the two or more transdifferentiation determinants comprise SPI1 and IKZF1.

37. The method of embodiment 36, wherein the method further comprises administering a nucleic acid encoding one or more of ID2, IRF8, BATF3, ATOX1, or BCL11A.

38. The method of any one of embodiments 22-37, wherein the method comprises administering to the glioblastoma three or more nucleic acids that increase and/or decrease expression of three or more transdifferentiation determinants.

39. The method of embodiment 38, wherein the three or more transdifferentiation determinants are selected from the group consisting of: SPI1, IKZF1, IRF8, ATOX1, BCL11A, ID2, BATF3, IKZF3, and GATA3.

40. The method of embodiment 39, wherein the three or more transdifferentiation determinants comprise at least SPI1 and IKZF1.

40.1. The method of any one of embodiments 22-37, wherein the method comprises administering to the glioblastoma four or more nucleic acids that increase and/or decrease expression of four or more different transdifferentiation determinants.

40.2. The method of any one of embodiments 22-37, wherein the method comprises administering to the glioblastoma five nucleic acids that increase and/or decrease expression of five different transdifferentiation determinants.

40.3 The method of embodiment 40.1 or 40.2, wherein at least two of the transdifferentiation determinants are selected from the group consisting of: SPI1, IKZF1, CTSZ, USF1, and PRKCB.

41. The method of any one of embodiments 1-40, wherein one or more nucleic acids comprise DNA, RNA or a combination thereof.

42. The method of any one of embodiments 1-41, wherein the one or more nucleic acids is provided in a viral vector.

43. The method of embodiment 42, wherein the viral vector is a lentivirus.

44. The method of embodiment 41, wherein the nucleic acids are provided on a plasmid, mRNA, or non-viral vector.

The method of embodiment 44, wherein the nucleic acids is administered by electroporation, direct injection, microprojectile bombardment, magneto-fection, sono-poration, photo-poration, or hydro-poration.

The method of embodiment 44, wherein the nucleic acid is provided in a nanoparticle, a lipid nanoparticle, a liposome, a lipoplex, a polyplex, a lipopolyplex, or other non-viral particle or complex.

The method of any one of embodiments 1-21, wherein the glioblastoma is in the brain or spinal cord.

The method of any one of embodiments 1-21, wherein expressing the one or more transdifferentiation determinants in the glioblastoma cells results in induction of an immune response against the glioblastoma.

Many modifications and other embodiments of the inventions set forth herein will come to mind to one skilled in the art to which the inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

### EXAMPLES

### Example 1. Genes Predicted to Facilitate Transdifferentiation of GBM Cells to Antigen Presenting Cells were Identified Using NETZEN (GeneRep-nSCORE)

Leveraging NETZEN, an integrated deep-learning and gene network-based ranking artificial intelligence (AI) platform for precision medicine developed by the inventors, transdifferentiation determinants for converting GBM cells in general to antigen presenting cells or GBM stem cells to antigen presenting cells were identified. Transdifferentiation determinants for both normal and pathologic conversions were identified.

### A) Mouse

TABLE 3
Top 20 Transdifferentiation Determinants for Transdifferentiation of
mouse high-grade glioma/GBM (GL261 cells) to DC and Macrophages, based
on prediction from in silico NETZEN (GeneRep-nSCORE) analysis.

| | |
|---|---|
| Transdifferentiation of GBM cells to | Transdifferentiation of GBM cells to |
| Dendritic-like Cells (DC-like) | Macrophage-like cells |

| Gene name | Abbreviation | Gene name | Abbreviation |
|---|---|---|---|
| IRF5 | I5 | SPI1 | dP |
| CBFA2T3 | C3 | IRF5 | I5 |
| IRF8 | I8 | IRF8 | I8 |
| ATOX1 | A | TFE3 | |
| SPI1 | S/dP/detaPEST/ | CEBPA | |
| | SP1/PU.1 | | |
| BCL11A | B | BCL11A | B |
| ID2 | I | CEBPB | |
| MYCL | M | SPIB | |
| BATF3 | B3 | POU2AF1 | |
| HHEX | | HELZ2 | |
| SPIB | | IKZF3 | |
| VAV1 | | MAFB | |
| ETV6 | | LMO2 | |
| MXD1 | | VAV1 | |
| ETV3 | | ARF2 | |
| LMO2 | | CBFA2T3 | C3 |
| AES | | MAZ | |
| GLMP | | PPARD | |
| CEBPA | | TAF10 | |
| MAZ | | ZFP384 | |

TABLE 4
Top 9 determinants for mouse GBM to antigen presenting cell
(dendritic cell-like) transdifferentiation, based on prediction
from in silico NETZEN (GeneRep-nSCORE) analysis.

| Gene | Function |
|---|---|
| IRF5 | Innate and Adaptive Immunity |
| CBFA2T3 | HSC Renewal |
| IRF8 | Myeloid Development |
| ATOX1 | Copper Chaperone |
| SPI1 | Myeloid Development |
| BCL11A | HSC Renewal |
| ID2 | Dendritic Cell Development |
| MYCL | Dendritic Cell Development |
| BATF3 | Dendritic Cell Development |

### B) Human Transdifferentiation Determinants

TABLE 5
Glioblastoma Mutliforme to antigen-presenting cell transdifferentiation
determinants - Top 20 predicted positive determinants as determined
by in silico NETZEN (GeneRep-nSCORE) analysis. Prediction
based on use of positive determinants.

| GBM-GSC to | GBM-GSC to | GBM cells lines | GBM cell lines |
|---|---|---|---|
| dendritic- | macrophage- | to dendritic- | to macrophage- |
| like cells | like cells | like cells | like cells |
| SPI1 | SPI1 | SPI1 | SPI1 |
| FOXP1 | CTSZ | CTSZ | CTSZ |
| CTSZ | TFEC | IKZF1 | HTATIP2 |
| HHEX | HTATIP2 | PRKCB | CREG1 |
| TFEC | FOXP1 | HTATIP2 | MMP14 |
| IKZF1 | CREG1 | TFEC | NR1H3 |
| HTATIP2 | TDP2 | USF1 | TFEC |
| KLF11 | IKZF1 | ZBTB34 | TDP2 |
| PRKCB | PRKCB | TDP2 | PRKCB |
| USF1 | CIR1 | ARID3A | SATB1 |
| TDP2 | NR1H3 | SATB1 | IKZF1 |
| BCL6 | KLF11 | FOXP1 | CIR1 |
| CREG1 | GNS | STAT6 | ARID3A |
| ZNF366 | MMP14 | CREG1 | NFE2L1 |
| LDB1 | HHEX | LMO2 | GNS |
| KMT2E | BASP1 | ZNF366 | ZBTB34 |
| CIITA | KMT2E | HHEX | NOTCH2 |
| ZFP91 | ATF5 | CIITA | MXD1 |
| ELF4 | NFE2L1 | KMT2E | USF2 |
| NCOA3 | IRF5 | NAB2 | MREG |

TABLE 6
Glioblastoma Mutliforme to antigen-presenting cell transdifferentiation
determinants - Top 20 predicted positive and negative determinants
as determined by in silico NETZEN (GeneRep-nSCORE) analysis. Prediction
based on combined use of positive and negative determinants. determinants.
“+” = positive regulator, “−” = negative regulator.

| | GBM-GSC to | | GBM-GSC to | | GBM cell lines | | GBM cell lines | |
|---|---|---|---|---|---|---|---|---|
| | dendritic- | | macrophage- | | to dendritic- | | to macrophage- | |
| Rank | like cells | | like cells | | like cells | | like cells | |
| | SPI1 | + | SPI1 | + | SPI1 | + | SPI1 | + |
| | CTSZ | + | ETS1 | − | CTSZ | + | ETS1 | − |
| | GATA3 | − | CTSZ | + | IKZF1 | + | CTSZ | + |
| | IKZF1 | + | TCF7 | − | USF1 | + | SATB1 | + |
| | HHEX | + | TFEC | + | PRKCB | + | RBPMS | − |
| | USF1 | + | CREG1 | + | GATA3 | − | PRKCB | + |
| | KLF11 | + | PRKCB | + | RBFOX2 | − | TCF7 | − |
| | RBFOX2 | − | RBPMS | − | SALL2 | − | MMP14 | + |
| | PRKCB | + | KLF11 | + | HTATIP2 | + | HTATIP2 | + |
| | TFEC | + | IKZF1 | + | TFEC | + | NR1H3 | + |
| | TCF7 | − | HTATIP2 | + | SATB1 | + | CREG1 | + |
| | SALL2 | − | ZNF507 | − | ZNF366 | + | CIR1 | + |
| | BCL6 | + | AEBP1 | − | ARID3A | + | IKZF1 | + |
| | LEF1 | − | CIR1 | + | LMO2 | + | TFEC | + |
| | ZNF366 | + | NR1H3 | + | LEF1 | − | ZNF507 | − |
| | HTATIP2 | + | SATB1 | + | ZNF74 | − | MXD1 | + |
| | RBI | + | HHEX | + | CREG1 | + | SALL2 | − |
| | BASP1 | + | MMP14 | + | ZBTB34 | + | ARID3A | + |
| | BCL11B | − | BASP1 | + | GTF2IRD1 | − | GNS | + |
| | CBFA2T3 | + | KLF12 | − | TCF7 | − | MREG | + |

TABLE 7
Glioblastoma Mutliforme to antigen-presenting cell transdifferentiation
determinants - Top 20 predicted positive determinants
as determined by in silico NETZEN (GeneRep-nSCORE) analysis.
Prediction based on combined use of positive and negative
determinants. determinants.

| | GBM-GSC to | GBM-GSC to | GBM cells line | GBM cell line |
|---|---|---|---|---|
| | dendritic- | macrophage- | to dendritic- | to macrophage- |
| Rank | like cells | like cells | like cells | like cells |
| | SPI1 | SPI1 | SPI1 | SPI1 |
| | CTSZ | CTSZ | CTSZ | CTSZ |
| | IKZF1 | TFEC | IKZF1 | SATB1 |
| | HHEX | CREG1 | USF1 | PRKCB |
| | USF1 | PRKCB | PRKCB | MMP14 |
| | KLF11 | KLF11 | HTATIP2 | HTATIP2 |
| | PRKCB | IKZF1 | TFEC | NR1H3 |
| | TFEC | HTATIP2 | SATB1 | CREG1 |
| | BCL6 | CIR1 | ZNF366 | CIR1 |
| | ZNF366 | NR1H3 | ARID3A | IKZF1 |
| | HTATIP2 | SATB1 | LMO2 | TFEC |
| | RBI | HHEX | CREG1 | MXD1 |
| | BASP1 | MMP14 | ZBTB34 | ARID3A |
| | CBFA2T3 | BASP1 | HHEX | GNS |
| | CIITA | GNS | CIITA | MREG |
| | TFEB | TDP2 | NFATC2 | TDP2 |
| | BID | MREG | PLAGL1 | NOTCH2 |
| | PLAGL1 | IRF8 | STAT6 | ZNF438 |
| | RUNX3 | GM2A | KLF11 | GM2A |
| | LDB1 | IRF5 | BID | LMO2 |

TABLE 8
Glioblastoma Mutliforme to antigen-presenting cell transdifferentiation
determinants - Top 20 predicted negative determinants
as determined by in silico NETZEN (GeneRep-nSCORE) analysis.
Prediction based on combined use of positive and negative
determinants. determinants.

| | GBM-GSC to | GBM-GSC to | GBM cells line | GBM cell line |
|---|---|---|---|---|
| | dendritic- | macrophage- | to dendritic- | to macrophage- |
| Rank | like cells | like cells | like cells | like cells |
| | GATA3 | ETS1 | GATA3 | ETS1 |
| | RBFOX2 | TCF7 | RBFOX2 | RBPMS |
| | TCF7 | RBPMS | SALL2 | TCF7 |
| | SALL2 | ZNF507 | LEF1 | ZNF507 |
| | LEF1 | AEBP1 | ZNF74 | SALL2 |
| | BCL11B | KLF12 | GTF2IRD1 | AEBP1 |
| | RORA | SALL2 | TCF7 | RBFOX2 |
| | TSHZ2 | RBFOX2 | PCGF2 | MYBL1 |
| | PTPN14 | ZNF483 | MYBL1 | ZEB1 |
| | GTF2IRD1 | LEF1 | ZNF280B | KLF4 |
| | KIF21A | PTPN14 | SOX13 | PTPN14 |
| | ETV5 | PDLIM1 | POU6F1 | TLE2 |
| | ATP8B2 | NFATC2 | PTPN14 | HOXB3 |
| | MAGED1 | GATA3 | BCL11B | ZNF483 |
| | CTTN | RORA | TSHZ2 | LOXL2 |
| | ZNF280B | TLE2 | ETV5 | PDLIM1 |
| | TCF19 | KLF4 | CTTN | LEF1 |
| | PCGF2 | PLCG1 | FOXP4 | KLF12 |
| | POU6F1 | PAWR | EGR1 | ZNF827 |
| | SOX13 | MAGED1 | ZNF507 | ZNF280B |

### Example 2. In Vitro Transdifferentiation of GBM Cells to Antigen Presenting Cells

GL261 glioblastoma cells or GL261 cells expressing a CD45-pro-GFP fusion protein were seeded into multi-well plates at 5×10⁴ cells/well on day 1. Cells were infected with lentivirus containing empty vector or an expression vector for expression of one or more GBM transdifferentiation genes on day 2. Cells were grown in RPMI1640 media with media change on days 1-4. Starting on day 4 or 5, cells were grown in RPMI640 media supplemented with GM-CSF (20 ng/mL) and IL4 (10 ng/mL). It is anticipated that GM-CSF and IL-4 would not be required in vivo wherein the cells naturally encounter conditions suited for growth. Media was replaced every 2 days. Cells were examined by flow cytometry on days 6, 9, and 13.

### Example 3. Transdifferentiation of GBM Cells to Antigen Presenting Cells: Granularity and CD45 Expression

As GBM cells became antigen presenting cells, they became more granular, reflected in an increase in side scatter (SCC) on a FACS plot. A nearly 7-fold increase in the cell fraction with high SCC was observed where GBM were modified to express SPI1, IRF8, BATF3 and ID2 compared to when they were infected with empty control virus (Table 9). The data show that expression of SPI1, IRF8, BATF3, and ID2 in GBM cells leads to the cells becoming more granular, which is indicative of transdifferentiation to antigen presenting cells. High SCC cells also exhibited much higher expression (˜10×) of CD45, a general marker for immune cells, compared to cells with low SCC. By day 9 GBM cells had transdifferentiated to antigen presenting cells as indicated by their become more granular, reflected in an increase in the side scatter (SCC) on a FACS plot.

TABLE 9
Side scatter in GBM cells following expression
of transdifferentiation determinants.

| | High side scatter | Low scatter |
|---|---|---|
| Treatment | (% cells) | (% cells) |
| pSF | 2.23 | 82 |
| SPI1 + IRF8 + BATF3 + ID2 | 15.1 | 42.2 |

### Example 4. Expression of MCHII in GBM Cells Treated with SPI1, IRF8, and BATF3

GBM cells were treating as described in example 2 to express either SPI1+IRF8+BATF3, or a control vector. On days 9 and 12, the cells were examined for the expression of MCHII and CD45 and side scatter. Expression of SPI1, IRF8, and BATF3 in GBM cells led to DC-like cells that expressed MCHII, CD45, and had increased side scatter. Expression of MCHII is a hallmark of antigen presenting cells. Increased side scatter was also observed in SPI1+IRF8+BATF3 treated cells. Expression of SPI1, IRF8, and BATF3 in GBM cells led to DC-like cells.

TABLE 10
MCHII expression in GBM cells following expression
of transdifferentiation determinants.

| | MHCII⁺/MHCII⁺CD11c⁺ | | CD45⁺/CD45⁺CD11b⁺ | |
|---|---|---|---|---|
| | (% cells) (DC-like) | | (% cells) (macrophage-like) | |
| Treatment | Days 9 | Day 12 | Days 9 | Day 12 |
| control | 0.36/0 | 0.69/0.10 | 0.36/0 | 0.44/0 |
| SPI1 + IRF8 + | 5.20/0.04 | 5.03/0.17 | 0.65/0 | 0.59/0 |
| BATF3 | | | | |

### Example 5. Expression of ID2 in GBM Cells Treated with SPI1, IRF8, and Batf3

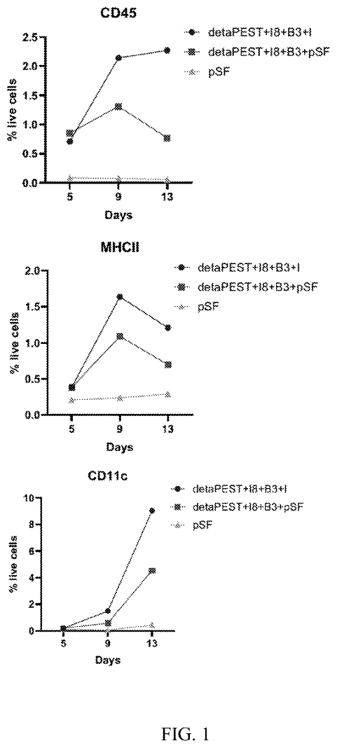

GBM cells were treated as described in example 2 to express either SPI1+IRF8+BATF3, SPI1+IRF8+BATF3+ID2, or control vectors. ID2 addition increased efficiency SPI1+IRF8+BATF3 to transdifferentiate GBM to DC (CD45+/MHCII+/CD11c+). Expression of SPI1+IRF8+BATF3 in GBM led to expression of MHCII and CD11c, indicating transdifferentiation of the GBM cells. Addition of ID2 led to further increase in the number of transdifferentiated cells (FIG. 1).

TABLE 11
Antigen presenting cell marker expression on in GBM cells on day
13 following expression of transdifferentiation determinants.

| | CD45⁺/ | CD24⁺/ | CD45+/MHCII⁺/ |
|---|---|---|---|
| | CD45⁺ | CD24⁺ | MHCII⁺ CD11c⁺ |
| Treatment | CD11c⁺ | CD45-proGFP⁺ | (DC-like) |
| control | 0.060/0 | 1.79/0.02 | 0/0 |
| SPI1 + IRF8 + | 0.41/0.36 | 4.70/0.25 | 3.75/2.73 |
| BATF3 | | | |
| SPI1 + IRF8 + | 1.46/0.81 | 2.89/0.18 | 4.33/3.54 |
| BATF3 + ID2 | | | |

### Example 6. Expression of SPI1, IRF8, and Batf3 in GBM Cells Results in Decreased Cell Growth Rate

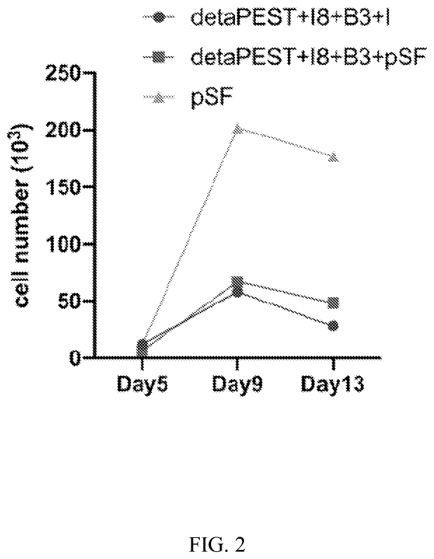

GBM cells were treated as described in example 2 to express either SPI1+IRF8+BATF3, SPI1+IRF8+BATF3+ID2, or control vectors. Cells expressing SPI1+IRF8+BATF3 or SPI1+IRF8+BATF3+ID2 had a much slower growth rate (FIG. 2). The decrease in growth rate is consistent with transdifferentiation. In addition to transdifferentiation, the delivery of SPI1+IRF8+BATF3 can also be used to reduce GBM growth.

### Example 7. Expression of CD45, CD11c, MHCII, and MHCI in GBM Cells Treated with SPI1, IRF8, BATF3, and ID2

GBM cells were treated as described in example 2 to express either SPI1+IRF8+BATF3+ID2, or control vectors. SPI1+IRF8+BATF3+ID2 expression in mouse GBM cells produced antigen-presenting cells with increases in CD45 expression.

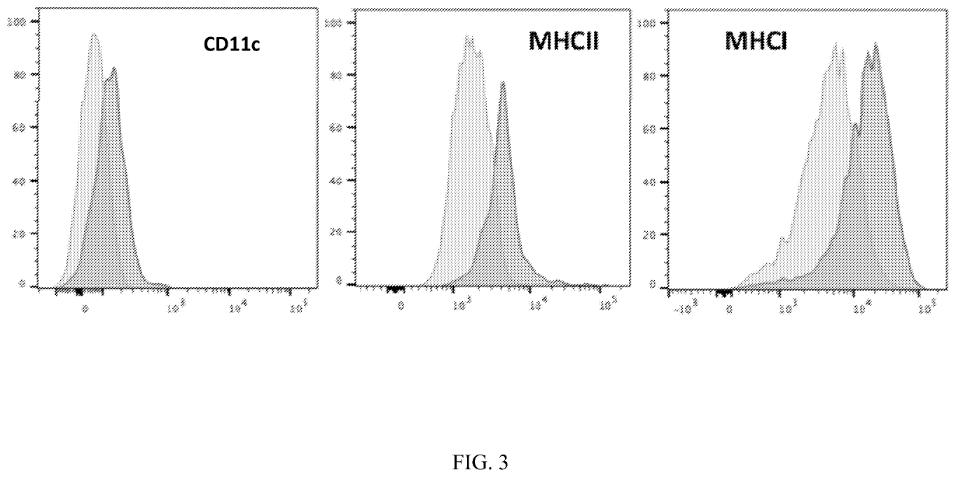

GBM cells treated to express SPI1+IRF8+BATF3+ID2 also had increased expression of CD11c, MHC1, and MHCII (FIG. 3). It is expected that the increase in MHCI and MHCII expression indicates the cells are capable of presenting antigens to CD4 and cross-presenting to CD8 T cells, a property that is mostly seen in DCs in vivo.

### Example 8. Efficient Reprogramming of GBM Cells to Immune Cells/DC-Like by Various Combinations of GBM to APC Transdifferentiation Various Transdifferentiation Determinants

GBM cells were treated as described in example 2 to express various combinations of SPI1, ID2, ATOX1, and/or BCL11A, or control vectors. Cells were then analyzed by FACS for MCHII and CD11c expression. The results indicate that each of the tested combinations leads to transdifferentiation of GBM cells to antigen presenting cells (Table 12). ATOX was not previously described to function in Immune cells, including DC. However, as predicted from the NETZEN (GeneRep-nSCORE) analysis, it functioned transdifferentiation of GBM to antigen presenting cells.

TABLE 12
Transdifferentiation of GBM cells to antigen presenting cells
using determinant combinations containing ATOX1 and/or BCL11A.

| | Percent | Percent |
|---|---|---|
| | MCHII⁺ cells | MCHII⁺CD11c⁺ cells |
| Determinants | in CD45⁺ cells | in CD45⁺ cells |
| Control vector | 0 | 0 |
| SPI1 + ID2 + | 3.31 | 2.90 |
| ATOX1 + BCL11A | | |
| SPI1 + ID2 + ATOX1 | 2.82 | 2.35 |
| SPI1 + ID2 + BCL11A | 2.99 | 1.99 |
| SPI1 + ATOX1 + BCL11A | 3.06 | 3.06 |
| ID2 + ATOX1 + BCL11A | 1.79 | 1.79 |

As shown in the examples above, mouse genes predicted to be transdifferentiation determinants using NETZEN (GeneRep-nSCORE), were found to transdifferentiate mouse glioblastoma cells. It is therefore expected that human genes predicted to be transdifferentiation determinants using NETZEN (GeneRep-nSCORE) will also function to transdifferentiate human glioblastoma cells when expressed in human glioblastoma cells. Further, the transdifferentiation determinants are expected to work better in vivo, where cells naturally experience growth factors and other conditions suited for cell growth.

### Example 9. Transdifferentiated GBM Cells are Able to Phagocytose

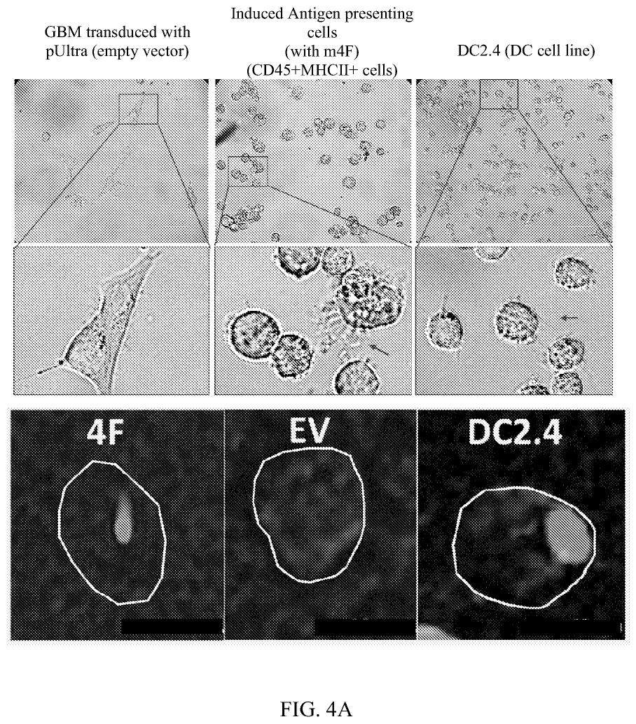

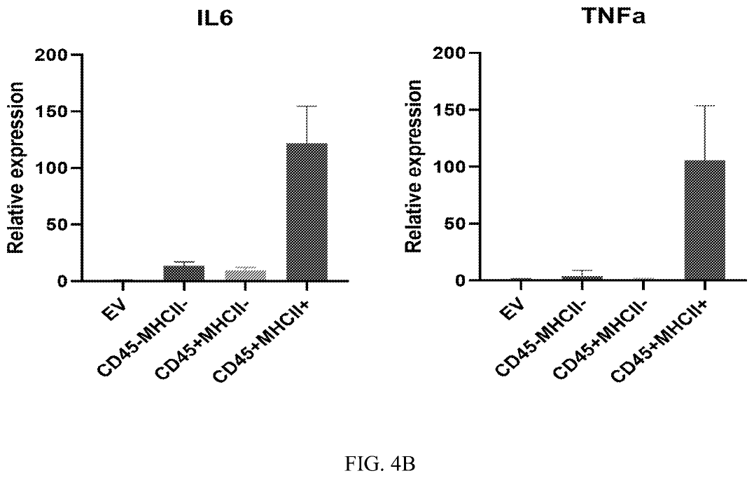

KR158 GBM cells reprogrammed with 4F (Spi1+IRF8+Batf3+Id2) produced antigen presenting cells that had DC-like features. The induced antigen presenting cells had morphology and dendrites (arrows) similar to dendritic cells (FIG. 4A). Induced DC functions were confirmed by their ability to phagocytose (ingest) pHrodo red Zymosan fungal particles (FIG. 4A). Zymosan fungal particles turn red once intracellularly. The data in FIG. 4 show that induced CD45⁺MHCII⁺ antigen presenting cells produced dendritic cell cytokines IL6 and TNFa while CD45⁻MHCII⁻ cells and CD45⁻MHCII⁺ cells did not (FIG. 4B).

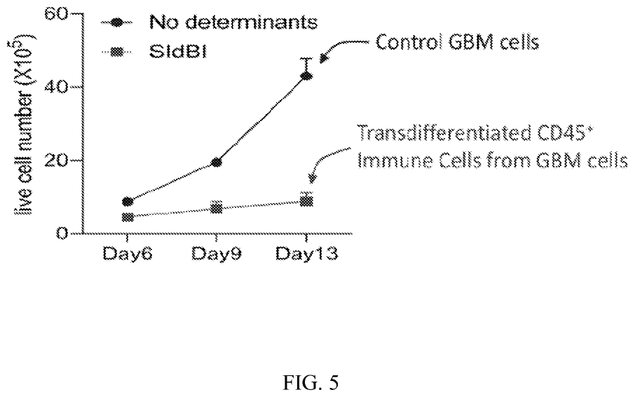

Induced immune cells (iCD45+ cells, which include iDC) lost their proliferative capacity when transdifferentiated from the highly proliferative GBM cells to become terminally differentiated cells. (FIG. 5).

### Example 10. KR158 GBM Cells Reprogrammed with Spi1+IRF8+Batf3+ID2 Become Immune-Like Cells with Better Antigen Presenting Capacity than Those Expressing Only Spi1 Alone and the Empty Vector Alone

KR158 GBM were transduced with transdifferentiation factors (dP=deltaPEST−Spi1; mF4=mouse 4 factor combination (Spi1+IRF8+Batf3+ID2) or empty virus control vector (control vehicle, pUltra) on day 1. Cells were re-seeded on day 4. Cells were transduced with Ova (which is processed and presented on MHC as SIINFEKL (SEQ ID NO: 1) on day 5.

On day 9, cells were co-cultured with OTI CD8⁺ or OTII CD4⁺ T cells (isolated from OT1 and OTII mice, respectively—these are transgenic mice that carry the SIINFEKL ova peptide antigen transgene such that all CD8 and CD4 T cells, respectively, are specific for SIINFEKL (SEQ ID NO: 1)). When specifically activated by DCs that present SIINFEKL (SEQ ID NO: 1) on their respective MHC, OT1 CD8⁺ and OTII CD4⁺ T cells become activated and produce interferon gamma (IFN-γ).

Flow cytometry was carried out on days 11 and 13.

Results: In cells transduced with control vehicle, 0.18% of cells presented SIINFEKL (SEQ ID NO: 1) on MHC. In cells transduced with deltaPEST−Spi1, 1.48% of cells presented SIINFEKL (SEQ ID NO: 1) on MHC (an 8 fold increase). In cells transduced with m4F, 5.61% of cells presented SIINFEKL (SEQ ID NO: 1) on MHC (a 31-fold increase over control). Ova-SIINFEKL was presented on H-2Kb MHC much more efficiently in cells reprogrammed with m4F and dP compared to control.

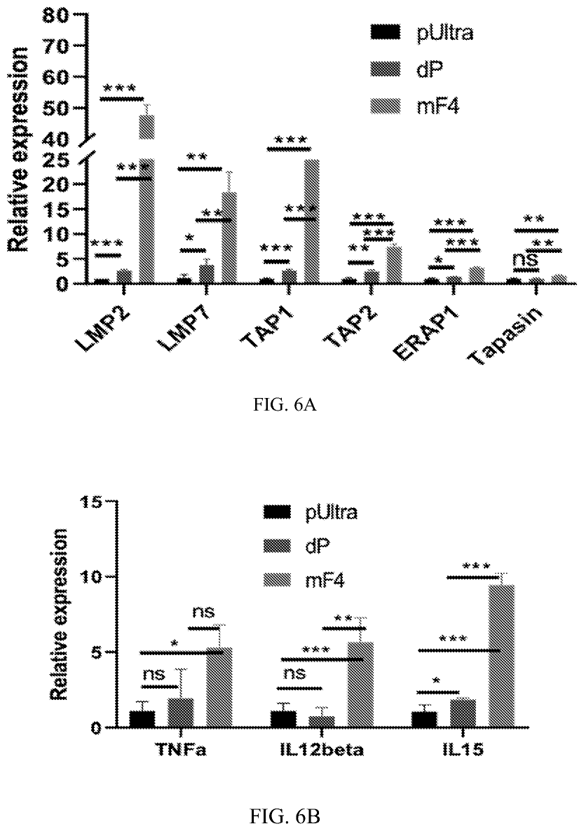

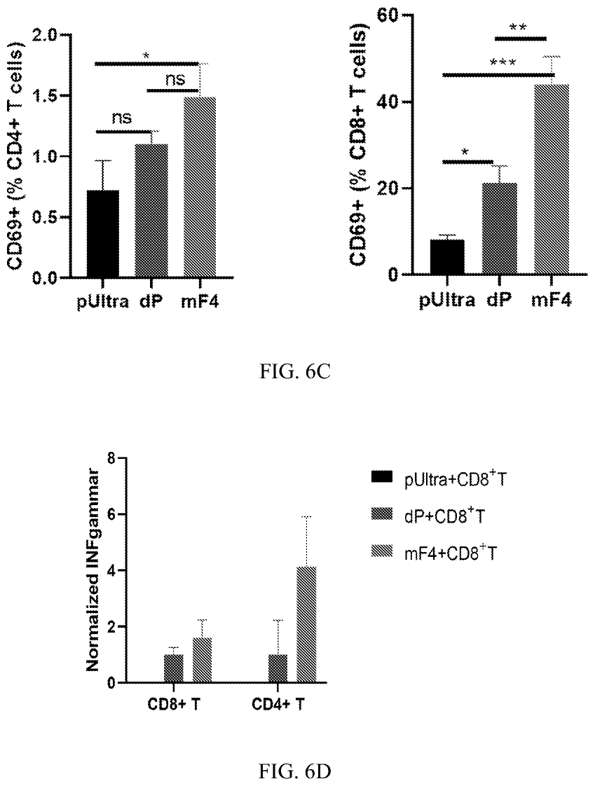

As shown in FIG. 6A, in cells transduced with either dP or m4F, expression of antigen processing and presentation machinery components was significantly upregulated compare to control cells, with mF4 cells exhibiting higher expression than dP cells.

DC-specific cytokines were also significantly upregulated in cells reprogrammed with 4F compared to control cells (FIG. 6B).

Antigen (SIINFEKL (SEQ ID NO: 1))-specific T cell activation (measured by the early activation marker CD69) was higher with induced DCs reprogrammed with 4F and dP compared to control cells (FIG. 6C).

Antigen (SIINFEKL (SEQ ID NO: 1))-specific T cell activation (INF-γ production) was more robust by induced DC-like cells reprogrammed with 4F or dP compared to control cells (FIG. 6D).

### Example 11. In Vivo Transdifferentiation of GBM Cells to Antigen Presenting Cells (e.g., Dendritic-Like Cells) to Induce Adaptive Immunity

On day 1, KR158-GBM-luc (KR-GBM) cells were injected into mice to form an intracranial tumor. On day 8, control virus or virus encoding one or more transdifferentiation genes was injected directly into the tumor: ((a) dP=deltaPEST−Spi1; (b) mF4=mouse 4 factor combination (Spi1+IRF8+Batf3+ID2); or (c) empty virus control vector (control vehicle, pUltra). On day 23, tumor infiltrating lymphocytes (TIL) and deep cervical lymph nodes (dcLN), which directly drain the brain, were collected for flow cytometry analysis. On days 23 and 37, tumor infiltrating lymphocytes (TIL) and dcLN, and peripheral blood mononuclear cells (PBMCs) and spleen were collected for flow cytometry analysis. CD8⁺ cells were isolated from the TILs and dcLN. The CD8⁺ TIL and dcLN CD8⁺ T cells were co-cultured with KR-GMB-luc. The cells were then assayed for activation of the CD8⁺ T cells and killing of the KR-GMB-luc cells. Activation of the CD8⁺ T cells and killing of the KR-GMB-luc indicates transdifferentiation of tumor KR-GMB-luc cells in the mice and induction of an immune response against the tumor in the mice. The CD8⁺ TIL and dcLN CD8⁺ T cells were also combined with autologous dendritic cells that had been pulsed with tumor lysate and tumor-specific T cell activation was measured.

Results: At 2 weeks after viral injection, a significant increase in central memory CD4 T cells (Tcm; CD44⁺CD62L⁺CD4⁺ T cells) as a percentage of CD4⁺ T cells in dcLNs was observed in animals receiving m4F compared to dP and pUltra.

At 2 weeks after viral injection, a significant increase in effector CD4 T cells (Tar; CD44⁺CD62L⁻CD4⁺ T cells) as a percentage of CD4⁺ T cells in dcLNs was observed in animals receiving m4F compared to dP and pUltra.

At 2 weeks after viral injection, a significant increase in central memory CD8 T cells (T_{cm}, CD44⁺CD62L⁺CD4⁺ T cells) as a percentage of CD8⁺ T cells in dcLNs was also observed in animals receiving m4F compared to dP and pUltra. The percentage of Tcm cells is expected to increase at later times.

CD8⁺ T from dcLNs were co-cultured with KR-GBM-luc cells. CD8⁺ T cells from animals with mF4 becomes more activated when cocultured with tumor cells compared CD8⁺ T cells from animals to dP and pUltra or in the absence of co-culturing.

### Example. 12. Transdifferentiation of Human Glioblastoma Cells

LN428, LN827, and LN308 cells are a human glioblastoma cell line.

The mouse experiments instructed that Spi1 expression was required for transdifferentiation of GBM cells to CD45⁺ immune cells and antigen-presenting cells or DC-like cells. In human GBM cells, Spi1 expression was also required for transdifferentiation of human GBM LN428 cells to CD45⁺ immune cells. Any combination lacking Spi1 did not transdifferentiate human LN428 GBM cells to CD45⁺ immune cells. The robustness of the identification that GATA3 needs to be repressed for transdifferentiation from human GBM cells to immune cells was tested by over pressing GATA3 with Spi1, which was predicted to antagonize the transdifferentiation effects of Spi1. Adding GATA3 overexpression to Spi1 overexpression reduced transdifferentiation of LN428 cells to CD45⁺ immune cells, consistent with this prediction. In contrast, Spi1 combined with Ikzf1 resulted in significantly increased transdifferentiation to CD45⁺ immune-like cells, indicating that Ikzf1 is not required for the transdifferention of LN428 cells to CD45⁺ immune cells but greatly enhances the transdifferentiation, and as such Ikzf1's role is likely not affected (increased or decreased) by GATA3. Indeed, the percentage of CD45⁺ immune cells was no better than EV control in LN428 cells transduced with Ikzf1 alone or Ikzf1+GATA3, and adding GATA3 overexpression did not increase the high rate of transdifferentiation by Spi1+Ikzf1 (Table 13).

TABLE 13
Percent CD45⁺ cells 7 days after transduction or human
LN428 cells with various transdifferentiation determinants.

% CD45⁺ cells at day 7

| Treatment | Experiment #1 | Experiment #2 |
|---|---|---|
| EV | 0.00929 | 0.086 |
| IKZF1 + EV | 0.13 | 0.11 |
| SPI1 + EV | 7.19 | 2.39 |
| EV + IKZF1 + GATA3 | 0.55 | 0.66 |
| SPI1 + EV + GATA3 | 1.1 | 1.27 |
| SPI1 + IKZF1 + EV | 21.5 | 17.2 |
| SPI1 + IKZF1 + GATA3 | 23.0 | 21.1 |

Transduction of LN428 cells with control empty vehicle virus expressing a puromycin resistance gene, followed by treatment with puromycin to eliminate un-transduced cells resulted in no CD45⁺ cells.

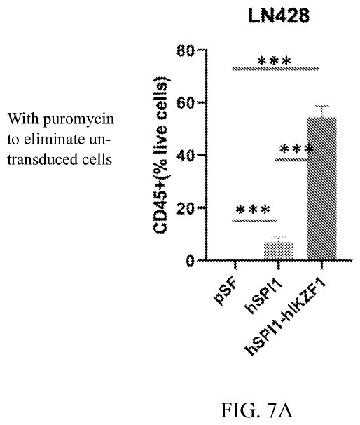

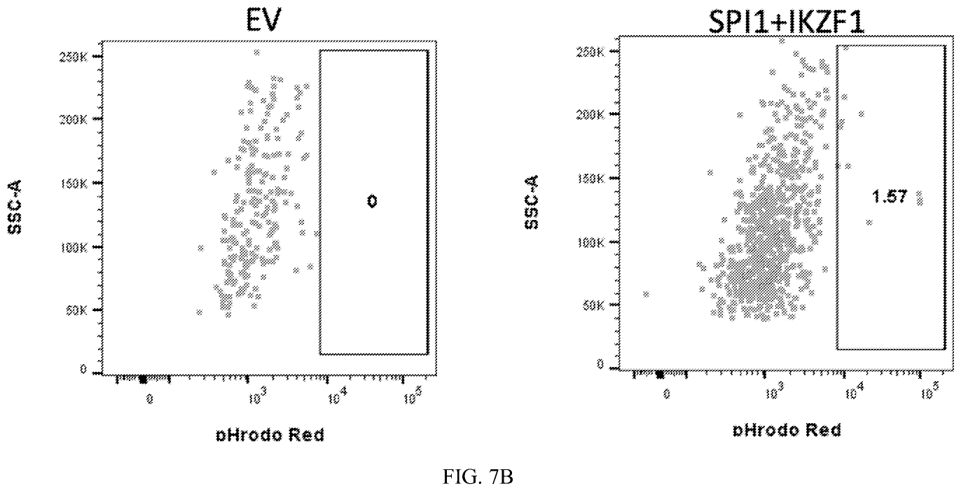

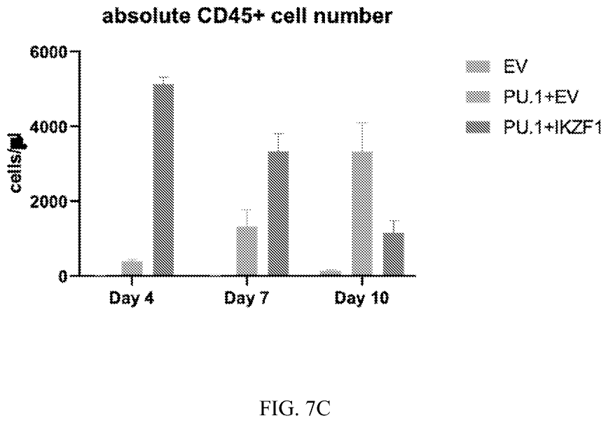

Transduction of LN428 cells with virus encoding Spi1+Ikzf1 and a puromycin resistance gene, followed by treatment with puromycin to eliminate un-transduced cells, resulted in 8-fold higher percent of live cells expressing CD45⁺ cells compared to Spi1 alone (FIG. 7A). 1.57% of CD45⁺ cells phagocytosed pHrodo Red particles (FIG. 7B).

Transduction of LN428 cells with virus encoding Spi1+Ikzf1 resulted in CD45⁺ cells that became reduced over time, indicating Spi1+Ikzf1-transdifferentiated CD45+ immune cells paused proliferation and died, compared to cells transdifferentiated with Spi1 alone continuing to grow over time (FIG. 7C).

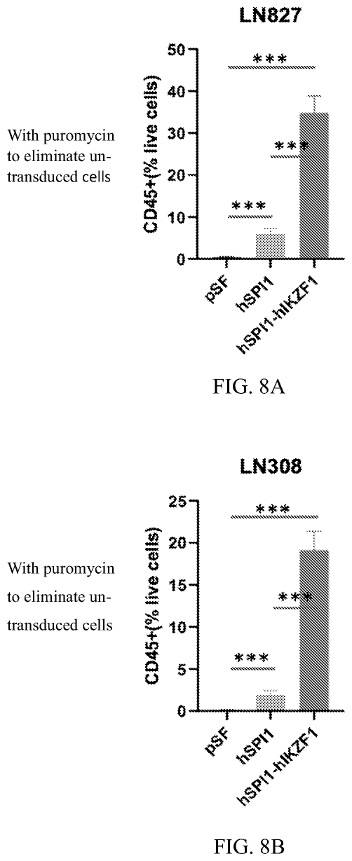

Transduction of LN827 GBM cells with virus encoding SPI1+IKZF1 and puromycin resistance, followed by puromycin treatment to eliminate un-transduced cells, was sufficient to transdifferentiate human GBM cells to CD45⁺ immune-like cells, resulting in 5-fold higher percentage of CD45⁺ immune cells compared to Spi1 alone (FIG. 8A).

Transduction of LN308 GBM cells with virus encoding SPI1+IKZF1 and puromycin resistance, followed by puromycin treatment to eliminate un-transduced cells, was sufficient to transdifferentiate human GBM cells to CD45⁺ immune-like cells, resulting in 9-fold higher percentage of CD45⁺ immune cells compared to Spi1 alone (FIG. 8B).

TABLE 14
Percent CD45⁺ and CD45⁺MHCII⁺ cells 7 days
after transduction or human LN308 GBM cells with various
transdifferentiation determinants.

| | % CD45⁺ cells | % CD45⁺MHCII⁺ cells |
|---|---|---|
| Treatment | at day 7 | at day 7 |
| EV (pSF) | 0.126 | 0.054 |
| pSF-hSPI1 | 1.659 | 0.029 |
| pSF-hSPI1 + IKZF1 | 18.14 | 0.34 |

hSpi1+hIKZF1 alone were sufficient to efficiently transdifferentiate human GBM cells to CD45⁺ immune-like cells. A small fraction of CD45⁺ immune-like cells acquired DC-like properties, including expressing MHCII and possessing phagocytic ability. Additional factors may increase efficiency of transdifferentiation of human GBM cells to antigen presenting cells (e.g., dendritic-like cells). Such additional factors may be selected from the top 20 ranked factors identified by NETZEN (see example 1). At alternative to empirically testing various combinations testing each combination separately, which is both laborious and time-consuming, we will instead perform comparative RNAseq expression profiling between human GBM cells and GBM cells partially reprogrammed with Spi1+Ikzf1 that express CD45 and human DC cells. We will also compare ChiPseq and methylation analyses between these cells. This approach will help identify the additional determinants that cooperate with Spi1+Ikzf1 to improve efficiently in transdifferentiating human GBM cells to antigen presenting cells.

As expected, expression of an exogenous GATA3 or RBFOX2 genes in glioblastoma cells did not improve transdifferentiation to CD45⁺ immune-like cells and antigen presenting cells. Because GATA3 and RBFOX2 are predicted to be negative regulators of human GBM cell transdifferentiation to antigen-presenting cells, it is expected that inhibition of GATA3 and/or RBFOX2 expression in glioblastoma cells would lead to improved transdifferentiation.

### Example 13. Single Cell RNASeq Analysis of Transdifferentiated Antigen Presenting Cells

RNASeq analysis can be used to show that CD45⁺MHCII⁺ cells produced by transdifferentiating GBM using mF4 or other combinations containing Spi1 are more dendritic cell-like based on their global gene expression profiles compared to those produced by Spi1 alone. RNASeq analysis can also be used to identify combinations that most efficiently transdifferentiation GBM cells to those that are the most similar to dendritic cells. Such combinations can then be used as in gene-therapy based immunotherapy.

Besides single cell RNASeq, serial ChiPseq to study the opening and closing of key chromatin regions during the reprogramming; and DNA methylation analysis to identify global regulatory changes in gene clusters as the reprogramming proceeds is performed. Results from these analyses will provide a more detailed mechanism of how the transdifferentiation occurs from GBM to antigen presenting cells, which may help reveal additional factors to improve reprogramming efficiency.

The following samples are analyzed using single cell RNAseq, ChipSeq and methylation analyses:
a) Negative control: EV Day 9 or GBM cells;
b) mF4 Day9 FACS for CD45⁺MHCII⁺ cells (GDM cells transduced with F4 and sorted for CD45⁺MHCII⁺ cells by FACS on day 9);
c) Spi1+Irf8+Bat3 Day9 FACS for CD45⁺MHCII⁺ cells;
d) Spi1+Irf8+Id2 Day9 FACS for CD45⁺MHCII⁺ cells;
e) Spi1+Bat3+Id2 Day9 FACS for CD45⁺MHCII⁺ cells;
f) Other combinations of mouse or human factors Day9 FACS for CD45⁺MHCII⁺ cells;
g) deltaPEST Day9 FACS for CD45⁺MHCII⁺ cells;
h) Positive control: mouse dendritic cells (60% dendritic cells, naïve); and/or
i) Positive control: mouse dendritic cells (60% dendritic cells, activated by GL261 and/or KR158 GBM cells).

### Example 14. Combinations of Transdifferentiation Determinants

Provided in the Table 15 are combinations of transdifferentiation determinants useful for transdifferentiating human GBM cells or GBM CSC to antigen presenting cells. “+” after the determinant indicates a positive regulator of transdifferentiation. “—” after the determinant indicates a negative regulator of transdifferentiation. For positive regulators, a nucleic acid that increases expression of the transdifferentiation determinant, such as a nucleic acid encoding the transdifferentiation determinant, is delivered to the GBM, GBM cell or GBM CSC. For native regulators, a nucleic acid that decreases expression of the transdifferentiation determinant, such as an expression inhibitor, is delivered to the GBM, GBM cell or GBM CSC.

TABLE 15
Combinations of transdifferentiation determinants.

| | Transdifferentiation Determinant | | | | | | | | | |
|---|---|---|---|---|---|---|---|---|---|---|
| combination | 1 | | 2 | | 3 | | 4 | | 5 | |
| 1 | SPI1 | + | IKZF1 | + | CTSZ | + | USF1 | + | | |
| 2 | SPI1 | + | IKZF1 | + | CTSZ | + | USF1 | + | PRKCB | + |
| 3 | SPI1 | + | IKZF1 | + | CTSZ | + | USF1 | + | TFEC | + |
| 4 | SPI1 | + | IKZF1 | + | CTSZ | + | PRKCB | + | | |
| 5 | SPI1 | + | IKZF1 | + | CTSZ | + | TFEC | + | | |
| 6 | SPI1 | + | IKZF1 | + | CTSZ | + | PRKCB | + | TFEC | + |
| 7 | SPI1 | + | IKZF1 | + | USF1 | + | PRKCB | + | TFEC | + |
| 8 | SPI1 | + | IKZF1 | + | PRKCB | + | TFEC | + | | |
| 9 | SPI1 | + | IKZF1 | + | PRKCB | + | TFEC | + | GATA3 | − |
| 10 | SPI1 | + | IKZF1 | + | PRKCB | + | TFEC | + | RBFOX2 | − |
| 11 | SPI1 | + | IKZF1 | + | PRKCB | + | TFEC | + | SALL2 | − |
| 12 | SPI1 | + | IKZF1 | + | GATA3 | − | | | | |
| 13 | SPI1 | + | IKZF1 | + | GATA3 | − | RBFOX2 | − | | |
| 14 | SPI1 | + | IKZF1 | + | GATA3 | − | RBFOX2 | − | SALL2 | − |
| 15 | SPI1 | + | IKZF1 | + | CTSZ | + | USF1 | + | GATA3 | − |
| 16 | SPI1 | + | IKZF1 | + | CTSZ | + | USF1 | + | RBFOX2 | − |
| 17 | SPI1 | + | IKZF1 | + | CTSZ | + | PRKCB | + | GATA3 | − |
| 18 | SPI1 | + | IKZF1 | + | CTSZ | + | PRKCB | + | RBFOX2 | − |
| 19 | SPI1 | + | IKZF1 | + | CTSZ | + | TFEC | + | GATA3 | − |
| 20 | SPI1 | + | IKZF1 | + | CTSZ | + | GATA3 | − | RBFOX2 | − |
| 21 | SPI1 | + | IKZF1 | + | USF1 | + | | | | |
| 22 | SPI1 | + | IKZF1 | + | USF1 | + | GATA3 | − | | |
| 23 | SPI1 | + | IKZF1 | + | USF1 | + | RBFOX2 | − | | |
| 24 | SPI1 | + | IKZF1 | + | USF1 | + | GATA3 | − | RBFOX2 | − |
| 25 | SPI1 | + | IKZF1 | + | USF1 | + | GATA3 | − | SALL2 | − |
| 26 | SPI1 | + | IKZF1 | + | PRKCB | + | | | | |
| 27 | SPI1 | + | IKZF1 | + | PRKCB | + | GATA3 | − | | |
| 28 | SPI1 | + | IKZF1 | + | PRKCB | + | RBFOX2 | − | | |
| 29 | SPI1 | + | IKZF1 | + | PRKCB | + | GATA3 | − | RBFOX2 | − |
| 30 | SPI1 | + | IKZF1 | + | PRKCB | + | GATA3 | − | SALL2 | − |
| 31 | SPI1 | + | IKZF1 | + | USF1 | + | PRKCB | + | | |
| 32 | SPI1 | + | IKZF1 | + | USF1 | + | PRKCB | + | GATA3 | − |
| 33 | SPI1 | + | IKZF1 | + | USF1 | + | PRKCB | + | RBFOX2 | − |
| 34 | SPI1 | + | IKZF1 | + | USF1 | + | PRKCB | + | SALL2 | − |
| 35 | SPI1 | + | IKZF1 | + | TFEC | + | | | | |
| 36 | SPI1 | + | IKZF1 | + | TFEC | + | GATA3 | − | | |
| 37 | SPI1 | + | IKZF1 | + | TFEC | + | RBFOX2 | − | | |
| 38 | SPI1 | + | IKZF1 | + | TFEC | + | GATA3 | − | RBFOX2 | − |
| 39 | SPI1 | + | IKZF1 | + | TFEC | + | SALL2 | − | | |
| 40 | SPI1 | + | IKZF1 | + | TFEC | + | GATA3 | − | SALL2 | − |
| 41 | SPI1 | + | IKZF1 | + | TFEC | + | RBFOX2 | − | SALL2 | − |
| 42 | SPI1 | + | CTSZ | + | USF1 | + | | | | |
| 43 | SPI1 | + | CTSZ | + | USF1 | + | GATA3 | − | | |
| 44 | SPI1 | + | CTSZ | + | USF1 | + | RBFOX2 | − | | |
| 45 | SPI1 | + | CTSZ | + | USF1 | + | GATA3 | − | RBFOX2 | − |
| 46 | SPI1 | + | CTSZ | + | USF1 | + | PRKCB | + | | |
| 47 | SPI1 | + | CTSZ | + | USF1 | + | PRKCB | + | GATA3 | − |
| 48 | SPI1 | + | CTSZ | + | USF1 | + | PRKCB | + | RBFOX2 | − |
| 49 | SPI1 | + | CTSZ | + | USF1 | + | PRKCB | + | SALL2 | − |
| 50 | SPI1 | + | CTSZ | + | USF1 | + | TFEC | + | | |
| 51 | SPI1 | + | CTSZ | + | USF1 | + | TFEC | + | GATA3 | − |
| 52 | SPI1 | + | CTSZ | + | USF1 | + | TFEC | + | RBFOX2 | − |
| 53 | SPI1 | + | CTSZ | + | USF1 | + | TFEC | + | SALL2 | − |
| 54 | SPI1 | + | USF1 | + | TFEC | + | | | | |
| 55 | SPI1 | + | USF1 | + | TFEC | + | GATA3 | − | RBBOX2 | − |
| 56 | SPI1 | + | USF1 | + | TFEC | + | GATA3 | − | SALL2 | − |
| 57 | SPI1 | + | USF1 | + | PRKCB | + | | | | |
| 58 | SPI1 | + | USF1 | + | PRKCB | + | GATA3 | − | | |
| 59 | SPI1 | + | USF1 | + | PRKCB | + | RBFOX2 | − | | |
| 60 | SPI1 | + | USF1 | + | PRKCB | + | GATA3 | − | RBFOX2 | − |
| 61 | SPI1 | + | USF1 | + | PRKCB | + | GATA3 | − | SALL2 | − |
| 62 | SPI1 | + | USF1 | + | PRKCB | + | RBFOX2 | − | SALL2 | − |
| 63 | SPI1 | + | USF1 | + | TFEC | + | | | | |
| 64 | SPI1 | + | USF1 | + | TFEC | + | GATA3 | − | | |
| 65 | SPI1 | + | USF1 | + | TFEC | + | RBFOX2 | − | | |
| 66 | SPI1 | + | USF1 | + | TFEC | + | GATA3 | − | RBFOX2 | − |
| 67 | SPI1 | + | USF1 | + | TFEC | + | GATA3 | − | SALL2 | − |
| 68 | SPI1 | + | USF1 | + | TFEC | + | RBFOX2 | − | SALL2 | − |
| 69 | SPI1 | + | PRKCB | + | TFEC | + | | | | |
| 70 | SPI1 | + | PRKCB | + | TFEC | + | GATA3 | − | | |
| 71 | SPI1 | + | PRKCB | + | TFEC | + | RBFOX2 | − | | |
| 72 | SPI1 | + | PRKCB | + | TFEC | + | GATA3 | − | RBFOX2 | − |
| 73 | SPI1 | + | PRKCB | + | TFEC | + | GATA3 | − | SALL2 | − |
| 74 | SPI1 | + | PRKCB | + | TFEC | + | RBFOX2 | − | SALL2 | − |

1. A method of treating glioblastoma comprising: administering to the glioblastoma in a subject one or more nucleic acids that increase and/or decrease expression of one or more glioblastoma to antigen presenting cell transdifferentiation determinants thereby increasing and/or decreasing expression of one or more of the transdifferentiation determinants in glioblastoma cells of the glioblastoma.

2. The method of claim 1, wherein the glioblastoma is a high-grade glioma or a glioblastoma multiforme.

3. The method of claim 1 or 2, wherein increasing and/or decreasing expression of the one or more transdifferentiation determinants in the glioblastoma cells results in transdifferentiation of one or more of the glioblastoma cells into antigen presenting cells.

4. The method of claim 1 or 2, wherein increasing and/or decreasing expression of the one or more transdifferentiation determinants in the glioblastoma cells results in reduced growth rate of the glioblastoma cells.

5. The method of any one of claim 1-4, wherein the one or more transdifferentiation determinants are selected from the list consisting of: SPI1, IKZF1, CTSZ, AEBP1, ARID3A, ATF5, ATP8B2, BASP1, BCL11B, BCL6, BID, CBFA2T3, CIITA, CIR1, CREG1, CTTN, EGR1, ELF4, ETS1, ETV5, FOXP1, FOXP4, GATA3, GM2A, GNS, GTF2IRD1, HHEX, HOXB3, HTATIP2, IRF5, IRF8, KIF21A, KLF4, KLF11, KLF12, KMT2E, LDB1, LEF1, LMO2, LOXL2, MAGED1, MMP14, MREG, MXD1, MYBL1, NAB2, NCOA3, NFATC2, NFE2L1, NOTCH2, NR1H3, PAWR, PCGF2, PDLIM1, PLAGL1, PLCG1, POU6F1, PRKCB, PTPN14, RB1, RBFOX2, RBPMS, RORA, RUNX3, SALL2, SATB1, SOX13, STAT6, TCF7, TCF19, TDP2, TFEB, TFEC, TLE2, TSHZ2, USF1, USF2, ZBTB34, ZEB1, ZFP91, ZNF74, ZNF280B, ZNF366, ZNF483, ZNF507, ZNF827, AES, ARF2, ATOX1, BATF3, BCL11A, CBFA2T3, CEBPA, CEBPB, PPARD, ETV3, ETV6, GLMP, HELZ2, ID2, IKZF3, MAFB, MAZ, MYCL, POU2AF1, SPIB, TAF10, TFE3, VAV1, and ZFP384.

6. The method of claim 5, wherein the one or more transdifferentiation determinants are selected from the list consisting of: IRF5, CBFA2T3, IRF8, ATOX1, SPI1, BCL11A, ID2, MYCL, BATF3, HHEX, SPIB, VAV1, ETV6, MXD1, ETV3, LMO2, AES, GLMP, CEBPA, MAZ, TFE3, CEBPB, POU2AF1, HELZ2, IKZF3, MAFB, ARF2, PPARD, TAF10, and ZFP384.

7. The method of claim 5, wherein the glioblastoma is a human glioblastoma and the one or more transdifferentiation determinants are selected from the list consisting of: SPI1, IKZF1, CTSZ, AEBP1, ARID3A, ATF5, ATP8B2, BASP1, BCL11B, BCL6, BID, CBFA2T3, CIITA, CIR1, CREG1, CTTN, EGR1, ELF4, ETS1, ETV5, FOXP1, FOXP4, GATA3, GM2A, GNS, GTF2IRD1, HHEX, HOXB3, HTATIP2, IRF5, IRF8, KIF21A, KLF4, KLF11, KLF12, KMT2E, LDB1, LEF1, LMO2, LOXL2, MAGED1, MMP14, MREG, MXD1, MYBL1, NAB2, NCOA3, NFATC2, NFE2L1, NOTCH2, NR1H3, PAWR, PCGF2, PDLIM1, PLAGL1, PLCG1, POU6F1, PRKCB, PTPN14, RB1, RBFOX2, RBPMS, RORA, RUNX3, SALL2, SATB1, SOX13, STAT6, TCF7, TCF19, TDP2, TFEB, TFEC, TLE2, TSHZ2, USF1, USF2, ZBTB34, ZEB1, ZFP91, ZNF74, ZNF280B, ZNF366, ZNF483, ZNF507, ZNF827.

8. The method of any one of claims 1-7, wherein the method comprises administering to the glioblastoma one or more nucleic acids that increase and/or decrease expression of one or more glioblastoma to antigen presenting cell transdifferentiation determinants thereby increasing and/or decreasing expression of one or more of the transdifferentiation determinants in glioblastoma cells of the glioblastoma.

9. The method of claim 8, wherein two or more transdifferentiation determinants are selected from the group consisting of: SPI1, IKZF1, IRF8, ATOX1, BCL11A, ID2, BATF3, IKZF3 and GATA3.

10. The method of claim 9 wherein the two or more transdifferentiation determinants comprise SPI1 and IKZF1.

11. The method of claim 10, wherein the method further comprises administering a nucleic acid encoding one or more of ID2, IRF8, BATF3, ATOX1, and BCL11A.

12. A method for transdifferentiation of glioblastoma cells into antigen presenting cells comprising: administering to the glioblastoma cells one or more nucleic acids that increase and/or decrease expression of one or more glioblastoma to antigen presenting cell transdifferentiation determinants thereby increasing and/or decreasing expression of one or more of the transdifferentiation determinants in the glioblastoma cells.

13. The method of claim 12, wherein the glioblastoma cell is a high-grade glioma cell or a glioblastoma multiforme cell.

14. The method of claim 12 or 13, wherein the antigen presenting cells are dendritic cell-like or macrophage-like.

15. The method of any one of claims 12-14, wherein the one or more transdifferentiation determinants are selected from the list consisting of: SPI1, IKZF1, CTSZ, AEBP1, ARID3A, ATF5, ATP8B2, BASP1, BCL11B, BCL6, BID, CBFA2T3, CIITA, CIR1, CREG1, CTTN, EGR1, ELF4, ETS1, ETV5, FOXP1, FOXP4, GATA3, GM2A, GNS, GTF2IRD1, HHEX, HOXB3, HTATIP2, IRF5, IRF8, KIF21A, KLF4, KLF11, KLF12, KMT2E, LDB1, LEF1, LMO2, LOXL2, MAGED1, MMP14, MREG, MXD1, MYBL1, NAB2, NCOA3, NFATC2, NFE2L1, NOTCH2, NR1H3, PAWR, PCGF2, PDLIM1, PLAGL1, PLCG1, POU6F1, PRKCB, PTPN14, RB1, RBFOX2, RBPMS, RORA, RUNX3, SALL2, SATB1, SOX13, STAT6, TCF7, TCF19, TDP2, TFEB, TFEC, TLE2, TSHZ2, USF1, USF2, ZBTB34, ZEB1, ZFP91, ZNF74, ZNF280B, ZNF366, ZNF483, ZNF507, ZNF827, AES, ARF2, ATOX1, BATF3, BCL11A, CBFA2T3, CEBPA, CEBPB, PPARD, ETV3, ETV6, GLMP, HELZ2, ID2, IKZF3, MAFB, MAZ, MYCL, POU2AF1, SPIB, TAF10, TFE3, VAV1, and ZFP384.

16. The method of claim 15, wherein the one or more transdifferentiation determinants are selected from the list consisting of: IRF5, CBFA2T3, IRF8, ATOX1, SPI1, BCL11A, ID2, MYCL, BATF3, HHEX, SPIB, VAV1, ETV6, MXD1, ETV3, LMO2, AES, GLMP, CEBPA, MAZ, TFE3, CEBPB, POU2AF1, HELZ2, IKZF3, MAFB, ARF2, PPARD, TAF10, and ZFP384.

17. The method of claim 15, wherein the glioblastoma is a human glioblastoma and the one or more transdifferentiation determinants are selected from the list consisting of: SPI1, IKZF1, CTSZ, AEBP1, ARID3A, ATF5, ATP8B2, BASP1, BCL11B, BCL6, BID, CBFA2T3, CIITA, CIR1, CREG1, CTTN, EGR1, ELF4, ETS1, ETV5, FOXP1, FOXP4, GATA3, GM2A, GNS, GTF2IRD1, HHEX, HOXB3, HTATIP2, IRF5, IRF8, KIF21A, KLF4, KLF11, KLF12, KMT2E, LDB1, LEF1, LMO2, LOXL2, MAGED1, MMP14, MREG, MXD1, MYBL1, NAB2, NCOA3, NFATC2, NFE2L1, NOTCH2, NR1H3, PAWR, PCGF2, PDLIM1, PLAGL1, PLCG1, POU6F1, PRKCB, PTPN14, RB1, RBFOX2, RBPMS, RORA, RUNX3, SALL2, SATB1, SOX13, STAT6, TCF7, TCF19, TDP2, TFEB, TFEC, TLE2, TSHZ2, USF1, USF2, ZBTB34, ZEB1, ZFP91, ZNF74, ZNF280B, ZNF366, ZNF483, ZNF507, ZNF827.

18. The method of any one of claims 12-17, wherein the method comprises administering to the glioblastoma two or more nucleic acids that increase and/or decrease expression of two or more transdifferentiation determinants.

19. The method of claim 18, wherein the two or more transdifferentiation determinants are selected from the group consisting of: SPI1, IKZF1, IRF8, ATOX1, BCL11A, ID2, BATF3, IKZF3, and GATA3.

20. The method of claim 19, wherein the two or more transdifferentiation determinants comprise SPI1 and IKZF1.

21. The method of claim 20, wherein the method further comprises administering a nucleic acid encoding one or more of ID2, IRF8, BATF3, ATOX1, and BCL11A.

22. The method of any one of claims 1-21, wherein the one or more nucleic acids is provided in a non-viral vector, plasmid, DNA vector, RNA vector, viral vector, or a lentiviral vector.

23. The method of any one of claims 1-11, wherein the glioblastoma is in the brain or spinal cord.

24. The method of any one of claims 1-11, wherein increasing and/or decreasing expression of the one or more transdifferentiation determinants in the glioblastoma cells results in induction of an immune response against the glioblastoma.

25. The method of any one of claims 1-24, wherein the transdifferentiation determinants are selected from the combinations of Table 15.
